(19) United States
(12) Patent Application Publication
(10) Pub. No.: US 2024/0033359 A1
(43) Pub. Date: Feb. 1, 2024

(54) COMPOSITIONS OF GUANYLYL CYCLASE C (GCC) ANTIGEN BINDING AGENTS AND METHODS OF USE THEREOF

(71) Applicants: TAKEDA PHARMACEUTICAL COMPANY LIMITED, Osaka (JP); CRESCENDO BIOLOGICS LTD, Cambridge (GB)

(72) Inventors: Gary SHAPIRO, Arlington, MA (US); Xingyue HE, Natick, MA (US); Mei Rosa NG, Charlestown, MA (US); Lorraine THOMPSON, Cambridge (GB); Elena DE JUAN FRANCO, Cambridge (GB); Steven VANCE, Chettisham, Ely (GB)

(21) Appl. No.: 18/256,578

(22) PCT Filed: Dec. 9, 2021
(86) PCT No.: PCT/IB2021/000852

§ 371 Date: Jun. 8, 2023

Related U.S. Application Data
(60) Provisional application No. 63/123,333, filed on Dec. 9, 2020.

(51) Int. Cl. A61K 39/00; C12N 5/0783; C12N 15/86; C07K 16/40; C07K 14/725; A61P 35/00

(52) U.S. Cl. CPC A61K 39/464454; C12N 5/0636; C12N 15/86; C07K 16/40; A61K 39/4611; A61K 39/4631; C07K 14/7051; A61P 35/00; C12N 2740/10043; C12N 2501/2302; C07K 2317/569; C07K 2317/92; C07K 2319/03

(57) ABSTRACT

Antigen binding agents (e.g., single domain antibodies) that bind guanylyl cyclase C (GCC) are disclosed. Nucleic acids, recombinant expression vectors, host cells, antigen binding fragments, and pharmaceutical compositions comprising these antigen binding agents and fragments thereof are also disclosed. The invention also provides therapeutic methods for utilizing the antibodies and antigen-binding molecules provided herein.

### CROSS-REFERENCE TO RELATED APPLICATIONS

This application claims priority to U.S. Provisional Patent Application Ser. No. 63/123,333, filed on Dec. 9, 2020, which is incorporated herein by reference in its entirety.

### SEQUENCE LISTING

The instant application contains a Sequence Listing, which was submitted electronically in ASCII format and is hereby incorporated by reference in its entirety. Said ASCII text file is incorporated herein by reference in its entirety, created on Nov. 18, 2021, is named “MIL-011 WO_SL” and is 33,391 bytes in size.

### BACKGROUND

Guanylyl cyclase C (GCC) is a transmembrane cell surface receptor that functions in the maintenance of intestinal fluid, electrolyte homeostasis and cell proliferation, see, e.g., Carrithers et al., Proc. Natl. Acad. Sci. USA 100:3018-3020 (2003). GCC is expressed at the mucosal cells lining the small intestine, large intestine and rectum (Carrithers et al., Dis Colon Rectum 39: 171-181 (1996)). GCC expression is maintained upon neoplastic transformation of intestinal epithelial cells, with expression in all primary and metastatic colorectal tumors (Carrithers et al., Dis Colon Rectum 39: 171-181 (1996); Buc et al. Eur J Cancer 41: 1618-1627 (2005); Carrithers et al., Gastroenterology 107: 1653-1661 (1994)). There is a need for novel and improved methods for targeting GCC.

### SUMMARY

Disruptions in GCC signaling pathways have been linked to numerous gastrointestinal disorders including colorectal cancer. The present invention provides, among other things, novel anti-GCC antigen binding molecules (e.g., single domain antibodies (sdAb)).

In one aspect, the present invention provides a guanylyl cyclase C (GCC) binding agent comprising a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16).

In one aspect, the present invention provides a guanylyl cyclase C (GCC) binding agent consisting of a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16).

In one aspect, the present invention provides a guanylyl cyclase C (GCC) binding agent comprising a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17).

In one aspect, the present invention provides a guanylyl cyclase C (GCC) binding agent consisting of a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17).

In one aspect, the present invention provides a guanylyl cyclase C (GCC) binding agent comprising a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18).

In one aspect, the present invention provides a guanylyl cyclase C (GCC) binding agent consisting of a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18).

In one aspect, the present invention provides a guanylyl cyclase C (GCC) binding agent comprising a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19).

In one aspect, the present invention provides a guanylyl cyclase C (GCC) binding agent consisting of a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19).

In one aspect, the present invention provides a guanylyl cyclase C (GCC) binding agent comprising a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

In one aspect, the present invention provides a guanylyl cyclase C (GCC) binding agent consisting of a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

In some embodiments, the GCC binding agent comprises an immunoglobulin heavy chain variable (V_{H}) region comprising an amino acid sequence that is at least 90% identical to SEQ ID NO: 1 or SEQ ID NO: 20.

In some embodiments, the GCC binding agent consists of an immunoglobulin heavy chain variable (V_{H}) region comprising an amino acid sequence that is at least 90° % identical to SEQ ID NO: 1 or SEQ ID NO: 20.

In some embodiments, the GCC binding agent comprises an immunoglobulin heavy chain variable (V_{H}) region comprising an amino acid sequence that is at least 90% identical to SEQ ID NO: 21.

In some embodiments, the GCC binding agent consists of an immunoglobulin heavy chain variable (V_{H}) region comprising an amino acid sequence that is at least 90% identical to SEQ ID NO: 21.

In some embodiments, the GCC binding agent comprises an immunoglobulin heavy chain variable (V_{H}) region comprising an amino acid sequence that is at least 90% identical to SEQ ID NO: 26.

In some embodiments, the GCC binding agent consists of an immunoglobulin heavy chain variable (V_{H}) region comprising an amino acid sequence that is at least 90% identical to SEQ ID NO: 26.

In some embodiments, the GCC binding agent comprises an immunoglobulin heavy chain variable (V_{H}) region comprising an amino acid sequence that is at least 90% identical to SEQ ID NO: 27.

In some embodiments, the GCC binding agent consists of an immunoglobulin heavy chain variable (V_{H}) region comprising an amino acid sequence that is at least 90% identical to SEQ ID NO: 27.

In some embodiments, the GCC binding agent comprises an immunoglobulin heavy chain variable (V_{H}) region comprising an amino acid sequence that is at least 90% identical to SEQ ID NO: 28.

In some embodiments, the GCC binding agent consists of an immunoglobulin heavy chain variable (V_{H}) region comprising an amino acid sequence that is at least 90% identical to SEQ ID NO: 28.

In one aspect, the present invention provides a guanylyl cyclase C (GCC) binding agent comprising an immunoglobulin heavy chain variable (V_{H}) region comprising an amino acid sequence that is at least 90% identical to SEQ ID NO: 1 or SEQ ID NO: 20.

In one aspect, the present invention provides a guanylyl cyclase C (GCC) binding agent consisting of an immunoglobulin heavy chain variable (V_{H}) region comprising an amino acid sequence that is at least 90% identical to SEQ ID NO: 1 or SEQ ID NO: 20.

In one aspect, the present invention provides a guanylyl cyclase C (GCC) binding agent comprising an immunoglobulin heavy chain variable (V_{H}) region comprising an amino acid sequence that is at least 90% identical to SEQ ID NO: 21.

In one aspect, the present invention provides a guanylyl cyclase C (GCC) binding agent consisting of an immunoglobulin heavy chain variable (V_{H}) region comprising an amino acid sequence that is at least 90% identical to SEQ ID NO: 21.

In one aspect, the present invention provides a guanylyl cyclase C (GCC) binding agent comprising an immunoglobulin heavy chain variable (V_{H}) region comprising an amino acid sequence that is at least 90% identical to SEQ ID NO: 26.

In one aspect, the present invention provides a guanylyl cyclase C (GCC) binding agent consisting of an immunoglobulin heavy chain variable (V_{H}) region comprising an amino acid sequence that is at least 900%6 identical to SEQ ID NO: 26.

In one aspect, the present invention provides a guanylyl cyclase C (GCC) binding agent comprising an immunoglobulin heavy chain variable (V_{H}) region comprising an amino acid sequence that is at least 90% identical to SEQ ID NO: 27.

In one aspect, the present invention provides a guanylyl cyclase C (GCC) binding agent consisting of an immunoglobulin heavy chain variable (V_{H}) region comprising an amino acid sequence that is at least 90% identical to SEQ ID NO: 27.

In one aspect, the present invention provides a guanylyl cyclase C (GCC) binding agent comprising an immunoglobulin heavy chain variable (V_{H}) region comprising an amino acid sequence that is at least 90% identical to SEQ ID NO: 28.

In one aspect, the present invention provides a guanylyl cyclase C (GCC) binding agent comprising or consisting of an immunoglobulin heavy chain variable (V_{H}) region comprising an amino acid sequence that is at least 90% identical to SEQ ID NO: 28.

In some embodiments, the GCC binding agent comprises a V_{H} region comprising an amino acid sequence that is at least 95% identical to any one of SEQ ID Nos: 1, 20, 21, 26, 27, or 28.

In some embodiments, the GCC binding agent consists of a V_{H} region comprising an amino acid sequence that is at least 95% identical to any one of SEQ ID Nos: 1, 20, 21, 26, 27, or 28.

In some embodiments, the GCC binding agent comprises a V_{H} region comprises an amino acid sequence that is identical to any one of SEQ ID NOs: 1, 20, 21, 26, 27, or 28.

In some embodiments, the GCC binding agent consists of a V_{H} region comprises an amino acid sequence that is identical to any one of SEQ ID NOs: 1, 20, 21, 26, 27, or 28.

In some embodiments, the GCC binding agent is selected from the group consisting of an IgA antibody, IgG antibody, IgE antibody, IgM antibody, bi- or multi-specific antibody, Fab fragment, Fab′ fragment, F(ab′)2 fragment, Fd′ fragment, Fd fragment, isolated CDRs or sets thereof; single-chain variable fragment (scFv), polypeptide-Fc fusion, single domain antibody (sdAb), camelid antibody; masked antibody, Small Modular ImmunoPharmaceuticals (“SMIPs™”), single chain, Tandem diabody, V_{H}Hs, Anticalin, Nanobody, humabody, minibodies, BiTE, ankyrin repeat protein, DARPIN, Avimer, DART, TCR-like antibody, Adnectin, Affilin, Trans-body; Affibody, TrimerX, MicroProtein, Fynomer, Centyrin; and KALBTTOR.

In some embodiments, the GCC binding agent is a single domain antibody (sdAb). In some embodiments, the GCC binding agent is a V_{H} single domain antibody.

In some embodiments, the GCC binding agent is a heavy chain only antibody.

In some embodiments, the GCC binding agent binds GCC with a KD between about 0.3 nanomolar (nM) and about 10 nM.

In some embodiments, the GCC binding agent binds GCC on target cells with an EC50 between about 0.5 nM and about 8 nM.

In one aspect, the present invention provides a method of treating a cancer comprising administering the GCC binding agent described herein to a subject in need of treatment.

In some embodiments, the cancer is selected from gastrointestinal cancer, colorectal cancer, colorectal adenocarcinoma, colorectal leiomyosarcoma, colorectal lymphoma, colorectal melanoma, a colorectal neuroendocrine tumor, metastatic colon cancer, stomach cancer, gastric adenocarcinoma, gastric lymphoma, gastric sarcoma, esophageal cancer, squamous cell carcinoma, adenocarcinoma of the esophagus, or pancreatic cancer.

In some embodiments, the cancer is a gastrointestinal cancer.

In some embodiments, the gastrointestinal cancer is colon cancer, colorectal cancer, stomach cancer, or esophageal cancer.

In one aspect, the present invention provides a pharmaceutical composition comprising a GCC binding agent and a pharmaceutically acceptable carrier, wherein the GCC binding agent comprises: a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16); a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17); a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18); a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19) or a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

In one aspect, the present invention provides a method of treating a cancer comprising administering an GCC binding agent to a subject in need of treatment, wherein the GCC binding agent comprises: a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16); a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17); a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18); a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19) or a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

In one aspect, the present invention provides a nucleic acid encoding a V_{H} amino acid sequence that is identical to any one of SEQ ID Nos: 1, 20, 21, 26, 27, or 28.

In one aspect, the present invention provides a vector comprising a nucleic acid encoding a V_{H} amino acid sequence that is identical to any one of SEQ ID Nos: 1, 20, 21, 26, 27, or 28.

In one aspect, the present invention provides an isolated cell comprising the vector comprising a nucleic acid encoding a V_{H} amino acid sequence that is identical to any one of SEQ ID Nos: 1, 20, 21, 26, 27, or 28.

In one aspect, the present invention provides an anti-guanylyl cyclase C (GCC) chimeric antigen receptor (CAR), wherein the anti-GCC CAR comprises an anti-GCC binding agent of any one of claims 1-10.

In one aspect, the present invention provides a method of inducing an immune response comprising contacting cells with an anti-guanylyl cyclase C (GCC) chimeric antigen receptor (CAR), wherein the anti-GCC CAR comprises an anti-GCC binding agent of any one of claims 1-10.

In one aspect, the present invention provides a method of inducing cytotoxicity comprising contacting cells with an anti-guanylyl cyclase C (GCC) chimeric antigen receptor (CAR), wherein the anti-GCC CAR comprises an anti-GCC binding agent of any one of claims 1-10.

In one aspect, the present invention provides a method of detecting the presence of cancer in a mammal, comprising: (a) contacting a sample comprising one or more cells from the mammal with the anti-GCC binding agent of any one of claims 1-10, thereby forming a complex, and (b) detecting the complex, wherein detection of the complex is indicative of the presence of cancer in the mammal.

In some embodiments, the contacting is in vitro or in vivo with respect to the mammal. In some embodiments, the contacting is in vitro.

It is to be understood that both the foregoing general description and the following detailed description are exemplary and explanatory only and are not restrictive of the invention, as claimed.

### BRIEF DESCRIPTION OF THE DRAWING

The Drawing included herein, which is comprised of the following Figures, is for illustration purposes only not for limitation.

FIGS. 1A and 1B depict exemplary chimeric antigen receptor (CAR) constructs.

FIGS. 2A-2D show exemplary anti-GCC CAR-T in vitro cytotoxicity performed using four tumor cell lines. HT29-GCC cells, a human colorectal cancer cell line HT29 engineered to stably express GCC) (FIG. 2A); HT29-VEC (vector control GCC-negative cell line) (FIG. 2B); and two tumor cell lines endogenously expressing GCC: GSU (FIG. 2C) and LS1034 (FIG. 2D). Bars represent mean+SD values from three technical replicates. Data are representative of >3 independent experiments performed with anti-GCC CAR T cells from >3 donors. CAR T cytotoxicity was determined in the absence of truncated EGFR (tEGFR).

FIGS. 3A-3D show exemplary anti-GCC CAR-T cells in vitro cytotoxicity performed using four tumor cell lines. HT29-GCC cells, a human colorectal cancer cell line HT29 engineered to stably express GCC) (FIG. 3A); HT29-VEC (vector control GCC-negative cell line) (FIG. 3B; and two tumor cell lines endogenously expressing GCC: GSU (FIG. 3C) and LS1034 (FIG. 3D). Bars represent mean+SD values from three technical replicates. Data are representative of >3 independent experiments performed with anti-GCC CAR T cells from >3 donors. CAR T cytotoxicity was determined in the presence of truncated EGFR (tEGFR).

FIGS. 4A-4D show exemplary IFN-g cytokine secretion by anti-GCC CAR-T cells co-cultured with GCC-expressing (HT29-GCC) (FIG. 4A), GSU (FIG. 4C), LS1034 (FIG. 4D) and GCC-negative (HT29-VEC, FIG. 4B) tumor cells in vitro. Secreted IFNg in the supernatant was detected using the Intellicyt QBeads Human PlexScreen kit (Sartorius, 90702). Bars represent mean+SD values from three technical replicates. Data are representative of >3 independent experiments performed with anti-GCC CAR T cells from >3 donors. Cytokine secretion was determined in the absence of truncated EGFR (tEGFR).

FIGS. 5A-5D show exemplary IFN-g cytokine secretion by anti-GCC CAR-T co-culture with GCC-expressing (HT29-GCC (FIG. 5A), GSU (FIG. 5C), LS1034 (FIG. 5D) and GCC-negative (HT29-VEC, FIG. 5B) tumor cells in vitro. Secreted IFNg in the supernatant was detected using the Intellicyt QBeads Human PlexScreen kit (Sartorius, 90702). Bars represent mean+SD values from three technical replicates. Data are representative of >3 independent experiments performed with anti-GCC CAR T cells from >3 donors. Cytokine secretion was determined in the presence of truncated EGFR (tEGFR).

FIGS. 6A-6D show exemplary IL-2 cytokine secretion by ant-GCC CAR-T co-culture with GCC-expressing (HT29-GCC (FIG. 6A), GSU (FIG. 6C), LS1034 (FIG. 6D) and GCC-negative (HT29-VEC, FIG. 6B) tumor cells in vitro. Secreted IFL-2 in the supernatant was detected using the Intellicyt QBeads Human PlexScreen kit (Sartorius, 90702). Bars represent mean+SD values from three technical replicates. Data are representative of >3 independent experiments performed with anti-GCC CAR T cells from >3 donors. Cytokine secretion was determined in the absence of truncated EGFR (tEGFR).

FIGS. 7A-7D show exemplary IL-2 cytokine secretion by anti-GCC CAR-T co-culture with GCC-(HT29-GCC (FIG. 7A), GSU (FIG. 7C), LS1034 (FIG. 7D) and GCC-negative (HT29-VEC, FIG. 7B) tumor cells in vitro. Secreted IFL-2 in the supernatant was detected using the Intellicyt QBeads Human PlexScreen kit (Sartorius, 90702). Bars represent mean+SD values from three technical replicates. Data are representative of >3 independent experiments performed with anti-GCC CAR T cells from >3 donors. Cytokine secretion was determined in the presence of truncated EGFR (tEGFR).

### DEFINITIONS

In order for the present invention to be more readily understood, certain terms are first defined below. Additional definitions for the following terms and other terms are set forth through the specification.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Administer: As used herein, “administering” a composition to a subject means to give, apply or bring the composition into contact with the subject. Administration can be accomplished by any of a number of routes, such as, for example, topical, oral, subcutaneous, intramuscular, intraperitoneal, intravenous, intrathecal, and intradermal.

Affinity: As used herein, the term “affinity” refers to the characteristics of a binding interaction between a binding moiety (e.g., an antigen binding agent (e.g., variable domain described herein) and a target (e.g., an antigen (e.g., GCC) and that indicates the strength of the binding interaction. In some embodiments, the measure of affinity is expressed as a dissociation constant (K_{D}). In some embodiments, a binding moiety has a high affinity for a target (e.g., a K_{D} of less than about 10⁻⁷ M, less than about 10⁻⁸ M, or less than about 10⁻⁹ M). In some embodiments, a binding moiety has a low affinity for a target (e.g., a K_{D} of higher than about 10⁻⁷ M, higher than about 10⁻⁶ M, higher than about 10⁻⁵ M, or higher than about 10⁻⁴ M).

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Autologous: As used herein, the term “autologous” is refers to any material derived from the same individual to whom it is later to be re-introduced into the individual.

Allogeneic “Allogeneic” refers to material derived from one individual administered to a different individual or individuals.

Antibody or Antigen Binding Agent: As used herein, the term “antibody” or “antigen binding agent” refers to a polypeptide that includes canonical immunoglobulin sequence elements sufficient to confer specific binding to a particular target antigen. Those skilled in the art will appreciate that the terms may be used herein interchangeably. In some embodiments, as used herein, the term “antibody” or “antigen binding agent” also refers to an “antibody fragment” or “antibody fragments” or “antigen binding portion”, which includes a portion of an intact antibody, such as, for example, the antigen-binding or variable region of an antibody. Examples of “antibody fragments” include Fab, Fab′, F(ab′)2, and Fv fragments; triabodies; tetrabodies; linear antibodies; single-chain antibody molecules; single domain antibodies; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (V_{H}) domain (located at the tips of the Y structure), followed by three constant domains: C_{H}1, C_{H}2, and the carboxy-terminal C_{H}3 (located at the base of the Y's stem). A short region, known as the “switch”, connects the heavy chain variable and constant regions. The “hinge” connects C_{H}2 and C_{H}3 domains to the rest of the antibody. Two disulfide bonds in this hinge region connect the two heavy chain polypeptides to one another in an intact antibody. Each light chain is comprised of two domains—an amino-terminal variable (V_{L}) domain, followed by a carboxy-terminal constant (C_{L}) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the C_{H}2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, and IgG4], and IgM) are defined based on the class of the utilized heavy chain sequences.

For purposes of the present invention, in certain embodiments, any polypeptide or complex of polypeptides that includes sufficient immunoglobulin domain sequences as found in natural antibodies can be referred to and/or used as an “antibody” or “antigen binding agent”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is polyclonal. In some embodiments, an antibody has constant region sequences that are characteristic of mouse, rabbit, primate, or human antibodies. In some embodiments, an antibody sequence elements are humanized, primatized, chimeric, etc., as is known in the art. Moreover, the term “antibody” or “antigen binding agent” as used herein, will be understood to encompass (unless otherwise stated or clear from context) can refer in appropriate embodiments to any of the art-known or developed constructs or formats for capturing antibody structural and functional features in alternative presentation. For example, in some embodiments, the terms can refer to bi- or other multi-specific (e.g., zybodies, etc.) antibodies, Small Modular ImmunoPharmaceuticals (“SMIPs™”), single chain antibodies, camelid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., polyethylene glycol, etc.]).

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Complementarity Determining Region (CDR): A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283: 1 156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding.

Unless stated otherwise, as used herein, CDR definitions are according to Kabat CDRs.

Effector functions: As used herein, the term “effector functions” refers to those biological activities attributable to an antigen binding agent described herein. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors; and B cell activation. “Reduced or minimized” antibody effector function means that which is reduced by at least 50% (alternatively 60%, 65%, 70%, 75%, 80%, 85%, 90%, 95%, 96%, 97%, 98%, or 99%6) from the wild type or unmodified antibody. The determination of antibody effector function is readily determinable and measurable by one of ordinary skill in the art. In some embodiments, the antibody effector functions of complement binding, complement dependent cytotoxicity and antibody dependent cytotoxicity are affected. In some embodiments, effector function is eliminated through a mutation in the constant region that eliminated glycosylation, e.g., “effector-less mutation.” In one aspect, the effector-less mutation is an N297A or DANA mutation (D265A+N297A) in the CH2 region. Shields et al., J. Biol. Chem. 276(9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in which result in an altered glycosylation pattern that is ineffective or less effective at promoting effector function (e.g., Shinkawa et al., J. Biol. Chem. 278 (5):3466-3473 (2003).

Antibody-dependent cell-mediated cytotaxicity or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95: 652-656 (1998).

Antigen: As used herein, the term “antigen”, refers to an agent that elicits an immune response; and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. In some embodiments of the disclosed compositions and methods, GCC protein is an antigen.

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can be assessed in any of a variety of contexts—including where interacting entities or moieties are studied in isolation or in the context of more complex systems (e.g., while covalently or otherwise associated with a carrier entity and/or in a biological system or cell). As used herein, “Kₐ” refers to an association rate of a particular binding moiety and a target to form a binding moiety/target complex. As used herein, “K_{d}” refers to a dissociation rate of a particular binding moiety/target complex. As used herein, “K_{D}” refers to a dissociation constant, which is obtained from the ratio of K_{d} to Kₐ(i.e., K_{d}/Kₐ) and is expressed as a molar concentration (M). K_{D} values can be determined using methods well established in the art, e.g., by using surface plasmon resonance, or using a biosensor system such as a Biacore® system.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Characteristic Portion: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity.

Codon-optimized: As used herein, a “codon-optimized” nucleic acid sequence refers to a nucleic acid sequence that has been altered such that translation of the nucleic acid sequence and expression of the resulting protein is improved optimized for a particular expression system. A “codon-optimized” nucleic acid sequence encodes the same protein as a non-optimized parental sequence upon which the “codon-optimized” nucleic acid sequence is based. For example, a nucleic acid sequence may be “codon-optimized” for expression in mammalian cells (e.g., CHO cells, human cells, mouse cells etc.), bacterial cells (e.g., E. coli), insect cells, yeast cells or plant cells.

Comparable: The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

Corresponding to: As used herein, the term “corresponding to” is often used to designate the position/identity of an amino acid residue in a polypeptide of interest. Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids.

Derived from: As used herein the phrase, a sequence “derived from” or “specific for a designated sequence” refers to a sequence that comprises a contiguous sequence of approximately at least 6 nucleotides or at least 2 amino acids, at least about 9 nucleotides or at least 3 amino acids, at least about 10-12 nucleotides or 4 amino acids, or at least about 15-21 nucleotides or 5-7 amino acids corresponding, i.e., identical or complementary to, e.g., a contiguous region of the designated sequence. In certain embodiments, the sequence comprises all of a designated nucleotide or amino acid sequence. The sequence may be complementary (in the case of a polynucleotide sequence) or identical to a sequence region that is unique to a particular sequence as determined by techniques known in the art. Regions from which sequences may be derived, include but are not limited to, regions encoding specific epitopes, regions encoding CDRs, regions encoding framework sequences, regions encoding constant domain regions, regions encoding variable domain regions, as well as non-translated and/or non-transcribed regions. The derived sequence will not necessarily be derived physically from the sequence of interest under study, but may be generated in any manner, including, but not limited to, chemical synthesis, replication, reverse transcription or transcription, that is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified or combined in ways known in the art to be consistent with the intended use. For example, a sequence may comprise two or more contiguous sequences which each comprise part of a designated sequence, and are interrupted with a region which is not identical to the designated sequence but is intended to represent a sequence derived from the designated sequence. With regard to antibody molecules, “derived therefrom” includes an antibody molecule which is functionally or structurally related to a comparison antibody, e.g., “derived therefrom” includes an antibody molecule having similar or substantially the same sequence or structure, e.g., having the same or similar CDRs, framework or variable regions. “Derived therefrom” for an antibody also includes residues, e.g., one or more, e.g., 2, 3, 4, 5, 6 or more residues, which may or may not be contiguous, but are defined or identified according to a numbering scheme or homology to general antibody structure or three-dimensional proximity, i.e., within a CDR or a framework region, of a comparison sequence. The term “derived therefrom” is not limited to physically derived therefrom but includes generation by any manner, e.g., by use of sequence information from a comparison antibody to design another antibody.

Determine: Many methodologies described herein include a step of “determining”. Those of ordinary skill in the art, reading the present specification, will appreciate that such “determining” can utilize any of a variety of techniques available to those skilled in the art, including for example specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source. In some embodiments, determining involves comparing one or more features of a sample or entity to a comparable reference.

Engineered: The term “engineered”, as used herein, describes a polynucleotide, polypeptide or a cell that has been designed or modified by man and/or whose existence and production require human intervention and/or activity. For example, an engineered cell that is intentionally designed to elicit a particular effect and that differs from the effect of naturally occurring cells of the same type. In some embodiments, an engineered cell expresses a chimeric antigen receptor described herein. Exemplary engineering methods are described in the detailed description and examples sections.

Epitope: As used herein, the term “epitope” includes any moiety that is specifically recognized by an immunoglobulin (e.g., antibody or receptor) binding component in whole or in part. In some embodiments, an epitope is comprised of a plurality of amino acids in an antigen. In some embodiments, such amino acid residues are surface-exposed when the antigen adopts a relevant three-dimensional conformation. In some embodiments, the amino acid residues are physically near to or contour with each other in space when the antigen adopts such a conformation. In some embodiments, at least some of the amino acids are physically separated from one another when the antigen adopts an alternative conformation (e.g., is linearized; e.g., a non-linear epitope).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: The term “expression” or “expressed”, when used in reference to a nucleic acid herein, refers to one or more of the following events: (1) production of an RNA transcript of a DNA template (e.g., by transcription); (2) processing of an RNA transcript (e.g., by splicing, editing, 5′ cap formation, and/or 3′ end formation); (3) translation of an RNA into a polypeptide; and/or (4) post-translational modification of a polypeptide.

Ex vivo: As used herein, the term “ex vivo” refers to events that occur in an external environment, e.g., outside a multi-cellular organism. In some embodiments, a cell or population of cells is modified outside of the body of a multi-cellular organism (e.g., a mammal such as a non-human primate or human being) to express a anti-GCC molecule described herein, prior to administration of such a cell or population of cells to a subject in need thereof.

Fusion protein: As used herein, the term “fusion protein” refers to a protein encoded by a nucleic acid sequence engineered from nucleic acid sequences encoding at least a portion of two different (e.g., heterologous) proteins. As persons of skill are no doubt aware, to create a fusion protein nucleic acid sequences are joined such that the resulting reading frame does not contain an internal stop codon. In some embodiments, fusion proteins as described herein include an influenza HA polypeptide or fragment thereof.

Guanylyl cyclase C (GCC): As used herein, “GCC,” also known as “STAR”, “GUC2C”, “GUCY2C” or “ST receptor” protein refers to mammalian GCC, preferably human GCC protein. Human GCC refers to the protein described in GenBank accession no.; NM-004963 and naturally occurring allelic protein variants thereof. Other variants are known in the art. See, e.g., accession number Ensp0000261170, Ensembl Database, European Bioinformatics Institute and Wellcome Trust Sanger Institute, US patent application number 20060035852; or GenBank accession number AAB 19934. Typically, a naturally occurring allelic variant has an amino acid sequence at least 95%, 97% or 99% identical to the GCC sequence of SEQ ID NO: 5. The transcript encodes a protein product of 1073 amino acids, and is described in GenBank accession no.: NM-004963. GCC protein is characterized as a transmembrane cell surface receptor protein, and is believed to play a critical role in the maintenance of intestinal fluid, electrolyte homeostasis and cell proliferation.

Host: The term “host” is used herein to refer to a system (e.g., a cell, organism, etc.) in which a polypeptide of interest is present. In some embodiments, a host is a system that expresses a particular polypeptide of interest.

Host cell: As used herein, the phrase “host cell” refers to a cell into which exogenous DNA (recombinant or otherwise) has been introduced. For example, host cells may be used to produce the polypeptides described herein by standard recombinant techniques. Persons of skill upon reading this disclosure will understand that such terms refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include any prokaryotic and eukaryotic cells suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, HEK293T, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6™ cell).

Immune response: As used herein, the term “immune response” refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro or ex vivo systems).

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured with human intervention. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced. In some embodiments, cells can be “isolated” (e.g., purified or separated) from other cells. For example, in some embodiments, genetically modified cells engineered to express a CAR described herein can be isolated from unmodified cells.

Nucleic acid: As used herein, the phrase “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, PA, 15^{th} Edition (1975), describes compositions and formulations suitable for pharmaceutical delivery of one or more therapeutic compositions, (e.g., a composition comprising a chimeric antigen receptor described herein), and additional pharmaceutical agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally. In some embodiments, the term “polypeptide” is used to refer to specific functional classes of polypeptides, such as, an antibody, chimeric antigen receptor, or costimulatory domain polypeptides, etc. For each such class, the present specification provides and/or the art is aware of several examples of amino acid sequences of known exemplary polypeptides within the class; in some embodiments, one or more such known polypeptides is/are reference polypeptides for the class. In such embodiments, the term “polypeptide” refers to any member of the class that shows sufficient sequence homology or identity with a relevant reference polypeptide that one skilled in the art would appreciate that it should be included in the class. In many embodiments, a member of the representative class also shares significant activity with the reference polypeptide. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region, often including a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

It is understood that the antibodies and antigen binding agents of the invention may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie, J U et al. Science 247:1306-1310 (1990) or Padlan et al. FASEB J. 9:133-139 (1995). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Recombinant: As used herein, the term “recombinant” is intended to refer to polypeptides (e.g., polypeptides as described herein) that are designed, engineered, prepared, expressed, created or isolated by recombinant means, such as polypeptides expressed using a recombinant expression vector transfected into a host cell, polypeptides isolated from a recombinant, combinatorial polypeptide library or polypeptides prepared, expressed, created or isolated by any other means that involves splicing selected sequence elements to one another. In some embodiments, one or more of such selected sequence elements is found in nature. In some embodiments, one or more of such selected sequence elements and/or combinations thereof is designed in silico. In some embodiments, one or more such selected sequence elements results from the combination of multiple (e.g., two or more) known sequence elements that are not naturally present in the same polypeptide (e.g., two epitopes from two separate HA polypeptides).

Reference: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest.

Single domain antibody: as used herein, the terms “single domain antibody (sdAb)”, “variable single domain” or “immunoglobulin single variable domain (ISV)” “single heavy chain variable domain (VH) antibody” refer to the single variable fragment of an antibody that binds to a target antigen and retains binding specificity to the antigen in the absence of light chain or other antibody fragments. These terms are used interchangeably herein. A sdAb is a single antigen-binding polypeptide having three complementary determining regions (CDRs). The sdAb alone is capable of binding to the antigen without pairing with a corresponding CDR-containing polypeptide. A VH single domain antibody refers to a single domain antibody that has a human heavy chain variable domain or a domain that is derived from a human heavy chain variable domain. In some cases, single-domain antibodies are engineered from camelid HCAbs, and their heavy chain variable domains of camelid HCAbs are referred to as “VHHs”. Some VHHs may also be known as Nanobodies. Camelid sdAb is one of the smallest known antigen-binding antibody fragments (see, e.g., Hamers-Casterman et al., Nature 363: 446-8 (1993); Greenberg et al., Nature 374: 168-73 (1995), Hassanzadeh-Ghassabeh et al., Nanomedicine (Lond), 8:1013-26 (2013)). A basic VH or VHH single domain antibody has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3. As explained below, some embodiments of the various aspects of the invention relate to a binding agent comprising a single heavy chain variable domain antibody/immunoglobulin heavy chain single variable domain which bind a GCC antigen in the absence of light chain.

Subject: As used herein, the term “subject” means any mammal, including humans. In certain embodiments of the present invention the subject is an adult, an adolescent or an infant. In some embodiments, terms “individual” or “patient” are used and are intended to be interchangeable with “subject”. Also contemplated by the present invention are the administration of the pharmaceutical compositions and/or performance of the methods of treatment in-utero. For example, a subject can be a patient (e.g., a human patient or a veterinary patient), having a cancer, (e.g., of gastrointestinal origin), a symptom of a cancer, in which at least some of the cells express GCC, or a predisposition toward a cancer, in which at least some of the cells express GCC. The term “non-human animals” of the invention includes all non-human vertebrates, e.g., non-human mammals and non-mammals, such as non-human primates, sheep, dog, cow, chickens, amphibians, reptiles, etc, unless otherwise noted.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the term “therapeutic agent” refers to an agent (e.g., an antigen binding agent) that has biological activity. The term is used herein to denote a chemical compound, a mixture of chemical compounds, a biological macromolecule, or an extract made from biological materials. In some embodiments, the therapeutic agent may be an anti-cancer agent or a chemotherapeutic agent. As used herein, the terms “anti-cancer agent” or “chemotherapeutic agent” refer to agents that have the functional property of inhibiting a development or progression of a neoplasm in a human, particularly a malignant (cancerous) lesion, such as a carcinoma, sarcoma, lymphoma, or leukemia. Inhibition of metastasis or angiogenesis is frequently a property of anti-cancer or chemotherapeutic agents. A chemotherapeutic agent may be a cytotoxic or cytostatic agent. The term “cytostatic agent” refers to an agent which inhibits or suppresses cell growth and/or multiplication of cells. In some embodiments, the therapeutic agent is a genetically modified cell or antibody. In some embodiments, the therapeutic agent is an anti-GCC CAR In some embodiments, the therapeutic agent is a cell (e.g., a population of cells) expressing a GCC CAR described herein.

Transformation: As used herein, refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, transfection, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time.

Treat or treatment: As used herein, the term “treat” or “treatment” is defined as the administration of an anti-GCC antigen binding agent (e.g., an anti-GCC antibody or fragment thereof, etc.) to a subject, e.g., a patient, or administration, e.g., by application, to an isolated tissue or cell from a subject which is returned to the subject. The anti-GCC antigen binding agent can be administered alone or in combination with a second agent. The treatment can be to cure, heal, alleviate, relieve, alter, remedy, ameliorate, palliate, improve or affect the disorder, the symptoms of the disorder or the predisposition toward the disorder, e.g., a cancer. While not wishing to be bound by theory, treating is believed to cause the inhibition, ablation, or killing of a cell in vitro or in vivo, or otherwise reducing capacity of a cell, e.g., an aberrant cell, to mediate a disorder, e.g., a disorder as described herein (e.g., a cancer).

Variable region or domain: As used herein, the terms “variable region” or “variable domain” of an antibody refers to the amino-terminal domains of the heavy or light chain of an antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies have a single heavy chain variable region.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

### DETAILED DESCRIPTION OF CERTAIN EMBODIMENTS

The present invention is based on the discovery of novel antigen binding agents that specifically bind guanylyl cyclase C (GCC) and their use in therapeutic methods. The present application provides anti-GCC single-domain antibodies (sdAb).

The present invention is not limited to particular methods, and experimental conditions described, as such methods and conditions may vary. It is also to be understood that the terminology used herein is for the purpose of describing particular embodiments only, and is not intended to be limiting unless indicated, since the scope of the present invention will be limited only by the appended claims.

### Guanylyl Cyclase C

Guanylyl cyclase C (GCC) (also known as STAR, ST Receptor, GUC2C, and GUCY2C) is a transmembrane cell surface receptor that functions in the maintenance of intestinal fluid, electrolyte homeostasis and cell proliferation (Carrithers et al., Proc Natl Acad Sci USA 100: 3018-3020 (2003); Mann et al., Biochem Biophys Res Commun 239: 463-466 (1997); Pitari et al., Proc Natl Acad Sci USA 100: 2695-2699 (2003)); GenBank Accession No. NM-004963, each of which is incorporated herein by reference). This function is mediated through binding of guanylin (Wiegand et al. FEBS Lett. 311:150-154 (1992)). GCC also is a receptor for heat-stable enterotoxin (ST, e.g., having an amino acid sequence of NTFYCCELCCNPACAGCY, SEQ ID NO: 29) which is a peptide produced by E. coli, as well as other infectious organisms (Rao, M. C. Ciba Found. Symp. 112:74-93 (1985); Knoop F. C. and Owens, M. J. Pharmacol. Toxicol. Methods 28:67-72 (1992)). Binding of ST to GCC activates a signal cascade that results in enteric disease, e.g., diarrhea. Nucleotide sequence for human GCC (GenBank Accession No. NM-004963). The amino acid sequence for human GCC (GenPept Accession No. NP-004954):

(SEQ ID NO: 5)
MKTLLLDLALWSLLFQPGWLSFSSQVSQNCHNGSYEISVLM
MGNSAFAEPLKNLEDAVNEGLEIVRGRLQNAGLNVTVNAT
FMYSDGLIHNSGDCRSSTCEGLDLLRKISNAQRMGCVLIG
PSCTYSTFQMYLDTELSYPMISAGSFGLSCDYKETLTRLM
SPARKLMYFLVNFWKINDLPFKTYSWSTSYVYKNGTETED
CFWYLNALEASVSYFSHELGFKVVLRQDKEFQDILMDHNR
KSNVIIMCGGPEFLYKLKGDRAVAEDIVIILVDLFNDQYF
EDNVTAPDYMKNVLVLTLSPGNSLLNSSFSRNLSPTKRDF
ALAYLNGILLFGHMLKIFLENGENITTPKFAHAFRNLTFE
GYDGPVTLDDWGDVDSTMVLLYTSVDTKKYKVLLTYDTHV
NKTYPVDMSPTFTWKNSKLPNDITGRGPQILMIAVFTLTG
AVVLLLLVALLMLRKYRKDYELRQKKWSHIPPENIFPLET
NETNHVSLKIDDDKRRDTIQRLRQCKYDKKRVILKDLKHN
DGNFTEKQKIELNKLLQIDYYNLTKFYGTVKLDTMIFGVI
EYCERGSLREVLNDTISYPDGTFMDWEFKISVLYDIAKGM
SYLHSSKTEVHGRLKSTNCVVDSRMVVKITDFGCNSILPP
KKDLWTAPEHLRQANISQKGDVYSYGIIAQEUILRKETFY
TLSCRDRNEKIFRVENSNGMKPFRPDLFLETAEEKELEVY
LLVKNCWEEDPEKRPDFKKIETTLAKIFGLFHDQKNESYM
DTLIRRLQLYSRNLEHLVEERTQLYKAERDRADRLNFMLL
PRLVVKSLKEKGFVEPELYEEVTIYFSDIVGFTTICKYST
PMEVVDMLNDIYKSFDHIVDHHDVYKVETIGDAYMVASGL
PKRNGNRHAIDIAKMALEILSFMGTFELEHLPGLPIWIRI
GVHSGPCAAGVVGIKMPRYCLFGDTVNTASRMESTGLPLR
IHVSGSTIAILKRTECQFLYEVRGETYLKGRGNETTYWLT
GMKDQKFNLPTPPTVENQQRLQAEFSDMIANSLOKRQAAG
IRSQKPRRVASYKKGTLEYLQLNTTDKESTYFF

The GCC protein has some generally accepted domains each of which contributes to the function of the GCC molecule. GCC functions include a signaling for directing the protein to the cell surface, an extracellular ligand binding, tyrosine kinase activity, and a guanylyl cyclase catalytic activity. In normal human tissues, GCC is expressed at the mucosal cells, e.g., at the apical brush border membranes, lining the small intestine, large intestine and rectum (Carrithers et al., Dis Colon Rectum 39: 171-181 (1996)). GCC expression is maintained upon neoplastic transformation of intestinal epithelial cells, with expression in all primary and metastatic colorectal tumors (Carrithers et al., Dis Colon Rectum 39: 171-181 (1996); Buc et al. Eur J Cancer 41: 1618-1627 (2005); Carrithers et al., Gastroenterology 107: 1653-1661 (1994)). Neoplastic cells from the stomach, esophagus and the gastroesophageal junction also express GCC (see, e.g., U.S. Pat. No. 6,767,704; Debruyne et al. Gastroenterology 130:1191-1206 (2006)). The tissue-specific expression and association with cancer, e.g., of gastrointestinal origin, (e.g., colon cancer, stomach cancer, or esophageal cancer), can be exploited for the use of GCC as a diagnostic marker for this disease (Carrithers et al., Dis Colon Rectum 39: 171-181 (1996); Buc et al. Eur J Cancer 41: 1618-1627 (2005)).

As a cell surface protein, GCC can also serve as a therapeutic target for receptor binding proteins such as antibodies or ligands. In normal intestinal tissue, GCC is expressed on the apical side of epithelial cell tight junctions that form an impermeable barrier between the luminal environment and vascular compartment (Almenoff et al., Mol Microbiol 8: 865-873); Guarino et al., Dig Dis Sci 32: 1017-1026 (1987)). As such, systemic intravenous administration of a GCC-binding protein therapeutic will have minimal effect on intestinal GCC receptors, while having access to neoplastic cells of the gastrointestinal system, including invasive or metastatic colon cancer cells, extraintestinal or metastatic colon tumors, esophageal tumors or stomach tumors, adenocarcinoma at the gastroesophageal junction. Additionally, GCC internalizes through receptor mediated endocytosis upon ligand binding (Buc et al. Eur J Cancer 41: 1618-1627 (2005); Urbanski et al., Biochem Biophys Acta 1245: 29-36 (1995)).

Polyclonal antibodies raised against the extracellular domain of GCC (Nandi et al. Protein Expr. Purif 8:151-159 (1996)) were able to inhibit the ST peptide binding to human and rat GCC and inhibit ST-mediated cGMP production by human GCC.

GCC has been characterized as a protein involved in cancers, including colon cancers. See also, Carrithers et al., Dis Colon Rectum 39: 171-181 (1996); Buc et al. Eur J Cancer 41: 1618-1627 (2005); Carrithers et al., Gastroenterology 107: 1653-1661 (1994); Urbanski et al., Biochem Biophys Acta 1245: 29-36 (1995).

Antigen binding molecule therapeutics directed to GCC described herein can be used to inhibit GCC-expressing cancerous cells. Anti-GCC antigen binding molecules of the invention can bind human GCC. In some embodiments, an anti-GCC antigen binding molecule of the invention can inhibit the binding of a ligand, e.g., guanylin or heat-stable enterotoxin to GCC.

### Antigen Binding Molecules

The present invention relates to anti-GCC antigen binding molecules. In some embodiments, anti-GCC molecules of the present inventions cause a cellular reaction upon binding to GCC on a GCC expressing cell to which it binds. In some embodiments, an anti-GCC antigen binding agent of the invention can block ligand binding to GCC.

The naturally occurring mammalian antibody structural unit is typified by a tetramer. Each tetramer is composed of two pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains can be classified as kappa and lambda light chains. Heavy chains can be classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site. Preferred isotypes for the anti-GCC antibody molecules are IgG immunoglobulins, which can be classified into four subclasses, IgG1, IgG2, IgG3 and IgG4, having different gamma heavy chains. Most therapeutic antibodies are human, chimeric, or humanized antibodies of the IgG1 type. In a particular embodiment, the anti-GCC antibody molecule has the IgG1 isotype.

The variable regions of each heavy and light chain pair form the antigen binding site. Thus, an intact IgG antibody has two binding sites which are the same. However, bifunctional or bispecific antibodies are artificial hybrid constructs which have two different heavy/light chain pairs, resulting in two different binding sites.

The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989). As used herein, CDRs are referred according to Kabat for each of the heavy (HCDR1, HCDR2, HCDR3) and light (LCDR1, LCDR2, LCDR3) chains.

An anti-GCC antibody molecule can comprise all, or an antigen binding subset of the CDRs or the heavy chain, of the antibodies described herein. Amino acid sequences of anti-GCC antigen binding agents described herein, including variable regions and CDRs, can be found in Tables 1-3.

Thus, in an embodiment the antibody molecule includes one or both of:
(a) one, two, three, or an antigen binding number of, light chain CDRs (LCDR1, LCDR2 and/or LCDR3) of a human antibody such as an antibody derived from a human hybridoma or a murine antibody (e.g., a light chain of an anti-GCC antibody described in US20180355062A1, which is incorporated by reference in it's entirety). In embodiments the CDR(s) may comprise an amino acid sequence of one or more or all of LCDR1-3 as follows: LCDR1, or modified LCDR1 wherein one to seven amino acids are conservatively substituted) LCDR2, or modified LCDR2 wherein one or two amino acids are conservatively substituted); or LCDR3, or modified LCDR3 wherein one or two amino acids are conservatively substituted; and
(b) one, two, three, or an antigen binding number of, heavy chain CDRs (HCDR1, HCDR2 and/or HCDR3) as described herein. In embodiments the CDR(s) may comprise an amino acid sequence of one or more or all of HCDR1-3 as follows. HCDR1, or modified HCDR1 wherein one or two amino acids are conservatively substituted; HCDR2, or modified HCDR2 wherein one to four amino acids are conservatively substituted, or HCDR3, or modified HCDR3 wherein one or two amino acids are conservatively substituted.

In some embodiments, an anti-GCC antibody molecule of the invention can draw antibody-dependent cellular cytotoxicity (ADCC) to a cell expressing GCC, e.g., a tumor cell. Antibodies with the IgG1 and IgG3 isotypes are useful for eliciting effector function in an antibody-dependent cytotoxic capacity, due to their ability to bind the Fc receptor. Antibodies with the IgG2 and IgG4 isotypes are useful to minimize an ADCC response because of their low ability to bind the Fc receptor. In related embodiments substitutions in the Fc region or changes in the glycosylation composition of an antibody, e.g., by growth in a modified eukaryotic cell line, can be made to enhance the ability of Fc receptors to recognize, bind, and/or mediate cytotoxicity of cells to which anti-GCC antibodies bind (see, e.g., U.S. Pat. Nos. 7,317,091, 5,624,821 and publications including WO 00/42072, Shields, et al. J. Biol. Chem. 276:6591-6604 (2001), Lazar et al. Proc. Natl. Acad. Sci. U.S.A. 103:4005-4010 (2006), Satoh et al. Expert Opin Biol. Ther. 6:1161-1173 (2006)). In certain embodiments, the antibody or antigen-binding fragment (e.g., antibody of human origin, human antibody) can include amino acid substitutions or replacements that alter or tailor function (e.g., effector function). For example, a constant region of human origin (e.g., 71 constant region, 72 constant region) can be designed to reduce complement activation and/or Fc receptor binding. (See, for example, U.S. Pat. No. 5,648,260 (Winter et al.), U.S. Pat. No. 5,624,821 (Winter et al.) and U.S. Pat. No. 5,834,597 (Tso et al.), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin. Additional anti-GCC antigen binding molecules are further described in U.S. Pat. No. 8,785,600 (Nam et al.), the entire teachings of which are incorporated herein by reference.

In still another embodiment, effector functions can also be altered by modulating the glycosylation pattern of the antibody. By altering is meant deleting one or more carbohydrate moieties found in the antibody, and/or adding one or more glycosylation sites that are not present in the antibody. For example, antibodies with enhanced ADCC activities with a mature carbohydrate structure that lacks fucose attached to an Fc region of the antibody are described in U.S. Patent Application Publication No. 2003/0157108 (Presta). See also U.S. Patent Application Publication No. 2004/0093621 (Kyowa Hakko Kogyo Co., Ltd). Glycofi has also developed yeast cell lines capable of producing specific glycoforms of antibodies.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which are engineered to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase M (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Humanized antibodies can also be made using a CDR-grafted approach. Techniques of generation of such humanized antibodies are known in the art. Generally, humanized antibodies are produced by obtaining nucleic acid sequences that encode the variable heavy and variable light sequences of an antibody that binds to GCC, identifying the complementary determining region or “CDR” in the variable heavy and variable light sequences and grafting the CDR nucleic acid sequences on to human framework nucleic acid sequences. (See, for example, U.S. Pat. Nos. 4,816,567 and 5,225,539). The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. J. Mol. Biol. 196:901-917 (1987)).

Anti-GCC antibody molecules described herein have the CDR amino acid sequences and nucleic acid sequences encoding CDRs listed in Tables 5 and 6. In some embodiments sequences from Tables 5 and 6 can be incorporated into molecules which recognize GCC for use in the therapeutic or diagnostic methods described herein. The human framework that is selected is one that is suitable for in vivo administration, meaning that it does not exhibit immunogenicity. For example, such a determination can be made by prior experience with in vivo usage of such antibodies and studies of amino acid similarities. A suitable framework region can be selected from an antibody of human origin having at least about 65% amino acid sequence identity, and preferably at least about 70%, 80%, 90% or 95% amino acid sequence identity over the length of the framework region within the amino acid sequence of the equivalent portion (e.g., framework region) of the donor antibody, e.g., an anti-GCC antibody molecule (e.g., 3G1). Amino acid sequence identity can be determined using a suitable amino acid sequence alignment algorithm, such as CLUSTAL W, using the default parameters. (Thompson J. D. et al., Nucleic Acids Res. 22:4673-4680 (1994).)

Once the CDRs and FRs of the cloned antibody that are to be humanized are identified, the amino acid sequences encoding the CDRs are identified and the corresponding nucleic acid sequences grafted on to selected human FRs. This can be done using known primers and linkers, the selection of which are known in the art. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen. After the CDRs are grafted onto selected human FRs, the resulting “humanized” variable heavy and variable light sequences are expressed to produce a humanized Fv or humanized antibody that binds to GCC. Preferably, the CDR-grafted (e.g., humanized) antibody binds a GCC protein with an affinity similar to, substantially the same as, or better than that of the donor antibody. Typically, the humanized variable heavy and light sequences are expressed as a fusion protein with human constant domain sequences so an intact antibody that binds to GCC is obtained. However, a humanized Fv antibody can be produced that does not contain the constant sequences.

Also within the scope of the invention are antibodies or fragments thereof as described herein, e.g. humanized antibodies, in which specific amino acids have been substituted, deleted or added in the CDR or framework regions. In particular, humanized antibodies can have amino acid substitutions in the framework region, such as to improve binding to the antigen. For example, a selected, small number of acceptor framework residues of the humanized immunoglobulin chain can be replaced by the corresponding donor amino acids. Locations of the substitutions include amino acid residues adjacent to the CDR, or which are capable of interacting with a CDR (see e.g., U.S. Pat. No. 5,585,089 or 5,859,205). The acceptor framework can be a mature human antibody framework sequence or a consensus sequence. As used herein, the term “consensus sequence” refers to the sequence found most frequently, or devised from the most common residues at each position in a sequence in a region among related family members. A number of human antibody consensus sequences are available, including consensus sequences for the different subgroups of human variable regions (see, Kabat, E. A., et al., Sequences of Proteins of Immunological Interest, Fifth Edition, U.S. Department of Health and Human Services, U.S. Government Printing Office (1991)). The Kabat database and its applications are freely available on line, e.g. via IgBLAST at the National Center for Biotechnology Information, Bethesda, Md. (also see, Johnson, G. and Wu, T. T., Nucleic Acids Research 29:205-206 (2001)).

In certain embodiments, the GCC antibody molecule is a human anti-GCC IgG1 antibody. Since such antibodies possess desired binding to the GCC molecule, any one of such antibodies can be readily isotype-switched to generate a human IgG4 isotype, for example, while still possessing the same variable region (which defines the antibody's specificity and affinity, to a certain extent). Accordingly, as antibody candidates are generated that meet desired “structural” attributes as discussed above, they can generally be provided with at least certain additional “functional” attributes that are desired through isotype switching.

In some aspects, the portion of a CAR composition of the invention that comprises an antibody fragment is humanized or optimised with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen.

In this way, FR residues can be selected and combined from the recipient and import sequences that the desired antibody or antibody fragment characteristic, such as increased affinity for the target antigen, is achieved. In general, the CDR residues are directly and most substantially involved in influencing antigen binding.

A humanized or optimised antibody or antibody fragment may retain a similar antigenic specificity as the original antibody, e.g., in the present invention, the ability to bind human GCC. In some embodiments, a humanized antibody or antibody fragment may have improved affinity and/or specificity of binding to human GCC.

In some embodiments, the anti-GCC antigen binding agent comprises one or more CDR sequences according provided in Table 1. In some embodiments, the anti-GCC antigen binding agent comprises a heavy chain variable region with a CDR 1 provided in Table 1. In some embodiments, the anti-GCC antigen binding agent comprises a heavy chain variable region with a CDR 2 provided in Table 1. In some embodiments, the anti-GCC antigen binding agent comprises a heavy chain variable region with a CDR 3 provided in Table 1. In some embodiments, the anti-GCC antigen binding agent comprises a heavy chain variable region with a CDR1, CDR2, and CDR3 provided in Table 1. In some embodiments, the anti-GCC antigen binding agent consists of a heavy chain variable region with a CDR1, CDR2, and CDR3 provided in Table 1. In some embodiments, the anti-GCC antigen binding agent comprises one or more CDR sequences provided in Table 1 wherein said CDR comprises 1, 2, or 3 amino acid substitutions. In one embodiment, said substitution does not adversely affect the binding of the binding agent to its target.

TABLE 1
Exemplary Anti-GCC CDR sequences
according to Kabat

| | HCDR 1 | HCDR 2 | HCDR 3 |
|---|---|---|---|
| V1 | HYYWS | RIYPSGSTSY | DRSTGWSEWN |
| | (SEQ ID | NPSLKS | SDL |
| | NO: 8) | (SEQ ID | (SEQ ID |
| | | NO: 11) | NO: 16) |
| V1-01 | HYYWS | RIYPSGSTSY | DRSTGWSEWN |
| | (SEQ ID | NPSLKS | SDL |
| | NO: 8) | (SEQ ID | (SEQ ID |
| | | NO: 11) | NO: 16) |
| V5 | RYWMS | KIRHDGGEKY | DYTRDV |
| | (SEQ ID | YVDSVKG | (SEQ ID |
| | NO: 9) | (SEQ ID | NO: 17) |
| | | NO: 12) | |
| V36 | RYWMT | KIKYDGSEKY | DYNKDY |
| | (SEQ ID | YADSVKG | (SEQ ID |
| | NO: 10) | (SEQ ID | NO: 18) |
| | | NO: 13) | |
| V48 | RYWMT | KIRHDGGEKY | DYNKDL |
| | (SEQ ID | YPDSVKG | (SEQ ID |
| | NO: 10) | (SEQ ID | NO: 19) |
| | | NO: 14) | |
| V51 | RYWMT | KIRHDGGEKY | DYNKDY |
| | (SEQ ID | YADSVKG | (SEQ ID |
| | NO: 10) | (SEQ ID | NO: 18) |
| | | NO: 15) | |

Anti-GCC antibodies that are not intact antibodies are also useful in this invention. In some embodiments, the anti-GCC antigen binding agent is a single domain antibody comprising a heavy chain variable region with a CDR1, CDR2, and CDR3 provided in Table 1. Such antibodies may be derived from any of the antibodies described above. Useful antibody molecules of this type include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region: (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; (vii) a single domain functional heavy chain antibody, which consists of a VHH domain (known as a nanobody) see e.g., Cortez-Retamozo, et al., Cancer Res. 64: 2853-2857 (2004), and references cited therein; and (vii) an isolated CDR, e.g., one or more isolated CDRs together with sufficient framework to provide an antigen binding fragment. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988); and Huston et al. Proc. Natl. Acad Sci. USA 85:5879-5883 (1988). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antibody fragments, such as Fv, F(ab′)₂ and Fab may be prepared by cleavage of the intact protein, e.g. by protease or chemical cleavage.

### Single-Domain Antibodies

Single-domain antibodies (sdAbs) are different from conventional 4-chain antibodies by having a single monomeric antibody variable domain. For example, camelids and sharks produce sdAbs named heavy chain-only antibodies (HcAbs), which naturally lack light chains. The antigen-binding fragment in each arm of the camelid heavy-chain only antibodies has a single heavy chain variable domain (VHH), which can have high affinity to an antigen without the aid of a light chain. Camelid VHH is known as the smallest functional antigen-binding fragment with a molecular weight of approximately 15 kD. In some embodiments, the antigen binding agents are single human heavy chain variable domain (VH) antibodies. Such binding molecules are also termed Humabody® and may be used interchangeably herein. Humabody® is a registered trademark of Crescendo Biologics Ltd.

One aspect of the present application provides isolated single-domain antibodies (referred herein as “anti-GCC sdAbs”) that specifically bind to GCC, such as human GCC. In some embodiments, the anti-GCC sdAb modulates GCC activity. In some embodiments, the anti-GCC sdAb is an antagonist antibody. Further provided are antigen-binding fragments derived from any one of the anti-GCC sdAbs described herein, and antigen binding proteins comprising any one of the anti-GCC sdAbs described herein. In some embodiments, the anti-GCC sdAb comprise one, two and/or three CDR sequences provided in Table 1. Exemplary anti-GCC sdAbs are listed in Table 2 and 3. In some embodiments, the anti-GCC sdAb comprises a variable heavy domain provided in Table 2 or Table 3. In some embodiments, the anti-GCC sdAb consists of a variable heavy domain provided in Table 2 or Table 3.

In some embodiments, some or all of the CDRs sequences, the VH domain or heavy chain, can be used in another antigen binding agent, e.g., in a CDR-grafted, humanized, or chimeric antibody molecule. Embodiments include an antibody molecule that comprises sufficient CDRs, e.g., all three CDRs from one of the above-referenced heavy chain variable region, to allow binding to cell surface GCC.

In some embodiments the CDRs, e.g., all of the HCDRs, are embedded in human or human derived framework region(s). Examples of human framework regions include human germline framework sequences, human germline sequences that have been affinity matured (either in vivo or in vitro), or synthetic human sequences, e.g., consensus sequences. In an embodiment the heavy chain framework is an IgG1 or IgG2 framework.

In some embodiments, the anti-GCC antigen binding agents of the present invention comprise a heavy chain variable region amino acid sequence provided in Table 2. In some embodiments, the anti-GCC antigen binding agents are single domain heavy chain only antibodies (e.g., antigen binding agents that do not comprise an immunoglobulin light chain).

TABLE 2
Exemplary Heavy Chain Variable
Region (VH) Amino Acid Sequences

| Description | VH Amino Acid Sequence |
|---|---|
| V1 | QVQLQESGPGLVKPSETLSLTCTVSGASIS |
| | HYYWSWFRQPAGKGLEWIGRIYPSGSTSYN |
| | PSLKSRVAMSVDTPKNQFSLNLSSVTAADT |
| | AVYYCARDRSTGWSEWNSDLWGRGTLVTVS |
| | S (SEQ ID NO: 1) |
| V1-01 | EVQLQESGPGLVKPSETLSLTCTVSGASIS |
| | HYYWSWFRQPAGKGLEWIGRIYPSGSTSYN |
| | PSLKSRVAMSVDTPKNQFSLKLSSVTAADT |
| | AVYYCARDRSTGWSEWNSDLWGRGTLVTVS |
| | S(SEQ ID NO: 20) |
| V5 | QVQLVESGGGLVQPGGSLRLSCTASGFTFS |
| | RYWMSWVRQAPGKGLEWVAKIRHDGGEKYY |
| | VDSVKGRFTISRDNAKNSLYLQMNSLRAED |
| | TAVYYCATDYTRDVWGQGTAVTVSS |
| | (SEQ ID NO: 21) |
| V36 | EVQLVESGGGLAQPGGSLRLSCAASGFTFS |
| | RYWMTWVRQAPGGRLEWVAKIKYDGSEKYY |
| | ADSVKGRFTISRDNAKNSLYLOMDSLRAED |
| | TAVYYCTRDYNKDYWGQGTLVTVSS |
| | (SEQ ID NO: 26) |
| V48 | EVQLVESGGGLVQPGGSLRLTCAASGFTFS |
| | RYWMTWVRQAPGKGLEWVAKIRHDGGEKYY |
| | PDSVKGRFTVSRDNAKNSLYLQMDNLRAED |
| | TAMYYCTRDYNKDLWGQGTLVTVSS |
| | (SEQ ID NO: 27) |
| V51 | EVQLVESGGGLVQPGGSLRLSCAASGFTFS |
| | RYWMTWVRQAPGKGLEWVAKIRHDGGEKYY |
| | ADSVKGRFTISRDNAKNSLYLQMNSLRAED |
| | TAVYYCTRDYNKDYWGQGTLVTVSS |
| | (SEQ ID NO: 28) |

In some embodiments, the anti-GCC antigen binding agents of the present invention comprise a heavy chain variable region amino acid sequence that is at least 90%, 91%, 92%, 93%, 94%, 95%, 96%, 97%, 98% or 99% identical to a VH sequence provided in Table 2. In some embodiments, the anti-GCC antigen binding agents of the present invention comprise a heavy chain variable region amino acid sequence as shown in Table 2 wherein said sequence comprises 1, 2, 3, 4, 5, 6, 7, 8, 9 or 10 amino acid substitutions. In one embodiment, the substitutions are outside the CDR regions. In some embodiments, the VH anti-GCC antigen binding agent (e.g., single domain antibody) comprises a leader sequence. In some embodiments, the VH anti-GCC antigen binding agent comprises a leader sequence comprising MKHLWFFLLLVAAPRWVLS (SEQ ID NO: 6), MELGLSWVFLVAILEGVQC (SEQ ID NO: 7) or MEFGLSWVFLVAIIKGVQC (SEQ ID NO: 2). In some embodiments, the VH anti-GCC antigen binding agent comprises a leader sequence comprising MALPVTALLLPLALLLHAARP (SEQ ID NO: 4)

In some embodiments, the anti-GCC antigen binding agents of the present invention comprise a heavy chain variable region amino acid sequence that is at least 90%, 91%, 92%, 93%, 94%, 95%, 96%, 97%, 98% or 99% identical to a VH sequence provided in Table 3. In some embodiments, the anti-GCC antigen binding agents of the present invention comprise a heavy chain variable region amino acid sequence as shown in Table 3 wherein said sequence comprises 1, 2, 3, 4, 5, 6, 7, 8, 9 or 10 amino acid substitutions. In one embodiment, the substitutions are outside the CDR regions. In some embodiments, the anti-GCC antigen binding agents of the present invention comprise a heavy chain variable region amino acid sequence that is identical to a VH sequence provided in Table 3.

In some embodiments, the VH anti-GCC antigen binding agent (e.g., single domain antibody) comprises a leader sequence provided that is at least 90%, 91%, 92%, 93%, 94%, 95%, 96%, 97%, 98% or 99% in Table 3. In some embodiments, the VH anti-GCC antigen binding agent (e.g., single domain antibody) comprises a leader sequence provided in Table 3.

TABLE 3
Exemplary Heavy Chain Variable Region
(VH) Amino Acid Sequences

| | Leader Sequence | VH sequence |
|---|---|---|
| V1 | MKHLWFFLLLVA | QVQLQESGPGLVKPSETLSL |
| | APRWVLS (SEQ | TCTVSGASISHYYWSWFRQP |
| | ID NO: 6) | AGKGLEWIGRIYPSGSTSYN |
| | | PSLKSRVAMSVDTPKNQFSL |
| | | NLSSVTAADTAVYYCARDRS |
| | | TGWSEWNSDLWGRGTLVTVS |
| | | S |
| | | (SEQ ID NO: 1) |
| V1-01 | MKHLWFFLLLVA | EVQLQESGPGLVKPSETLSL |
| | APRWVLS (SEQ | TCTVSGASISHYYWSWFRQP |
| | ID NO: 6) | AGKGLEWIGRIYPSGSTSYN |
| | | PSLKSRVAMSVDTPKNQFSL |
| | | KLSSVTAADTAVYYCARDRS |
| | | TGWSEWNSDLWGRGTLVTVS |
| | | S |
| | | (SEQ ID NO: 20) |
| V5 | MELGLSWVFLVAI | QVQLVESGGGLVQPGGSLRL |
| | LEGVQC | SCTASGFTFSRYWMSWVRQA |
| | (SEQ ID NO: 7) | PGKGLEWVAKIRHDGGEKYY |
| | | VDSVKGRFTISRDNAKNSLY |
| | | LQMNSLRAEDTAVYYCATDY |
| | | TRDVWGQGTAVTVSS |
| | | (SEQ ID NO: 21) |
| V8 | MELGLSWVFLVAI | QVQLVESGGGLVQPGGSLRL |
| | LEGVQC | SCAASGFTFSRYWMSWVRQA |
| | (SEQ ID NO: 7) | PGKGLEWVAKIKYDGSEKYY |
| | | VDSVKGRFTISRDNAKNSVY |
| | | LQMNSLRAEDTGVYYCATDF |
| | | TRDVWGQGTTVTVSS |
| | | (SEQ ID NO: 22) |
| V9 | MELGLSWVFLVAI | EVQLVESGGGLVQPGGSLRL |
| | LEGVQC | SCAASGFTFSRYWMTWVRQA |
| | (SEQ ID NO: 7) | PGRGLEWVAKIRYDGGEKYY |
| | | VDSVKGRFTISRDNAKNSLY |
| | | LQMNSLRAEDTAVYYCATDF |
| | | TRDVWGQGTTVTVSS |
| | | (SEQ ID NO: 23) |
| V30 | MELGLSWVFLVAI | QVQLVESGGGLVQPGGSLRL |
| | LEGVQC | SCAASGFNFGRYWMSWVRQA |
| | (SEQ ID NO: 7) | PGKGREWVAKIKYDGSEKYY |
| | | VDSVKGRFTISRDNAKNSLY |
| | | LQMNSLRAEDTAVYYCATDF |
| | | TRDVWGQGTTVTVSS |
| | | (SEQ ID NO: 24) |
| V31 | MEFGLSWVFLVAI | QVQLVESGGGVVRPGGSLRL |
| | IKGVQC (SEQ ID | SCAASGFTFSRYWMSWVRQA |
| | NO: 2) | PGKGREWVAKIKYDGSEKYY |
| | | ADSVKGRFTISRDNAKNSLY |
| | | LQMNSLRADDTAVYYCATDF |
| | | TRDVWGQGTTVTVSS |
| | | (SEQ ID NO: 25) |
| V36 | MELGLSWVFLVAI | EVQLVESGGGLAQPGGSLRL |
| | LEGVQC | SCAASGFTFSRYWMTWVRQA |
| | (SEQ ID NO: 7) | PGGRLEWVAKIKYDGSEKYY |
| | | ADSVKGRFTISRDNAKNSLY |
| | | LQMDSLRAEDTAVYYCTRDY |
| | | NKDYWGQGTLVTVSS |
| | | (SEQ ID NO: 26) |
| V48 | MELGLSWVFLVAI | EVQLVESGGGLVQPGGSURL |
| | LEGVQC | TCAASGFTFSRYWMTWVRQA |
| | (SEQ ID NO: 7) | PGKGLEWVAKIRHDGGEKYY |
| | | PDSVKGRFTVSRDNAKNSLY |
| | | LQMDNLRAEDTAMYYCTRDY |
| | | NKDLWGQGTLVTVSS |
| | | (SEQ ID NO: 27) |
| V51 | MELGLSWVFLVAI | EVQLVESGGGLVQPGGSLRL |
| | LEGVQC | SCAASGFTFSRYWMTWVRQA |
| | (SEQ ID NO: 7) | PGKGLEWVAKIRHDGGEKYY |
| | | ADSVKGRFTISRDNAKNSLY |
| | | LQMNSLRAEDTAVYYCTRDY |
| | | NKDYWGQGTLVTVSS |
| | | (SEQ ID NO: 28) |

Antibody fragments for in vivo therapeutic or diagnostic use can benefit from modifications which improve their serum half-lives. Suitable organic moieties intended to increase the in vivo serum half-life of the antibody can include one, two or more linear or branched moiety selected from a hydrophilic polymeric group (e.g., a linear or a branched polymer (e.g., a polyalkane glycol such as polyethylene glycol, monomethoxy-polyethylene glycol and the like), a carbohydrate (e.g., a dextran, a cellulose, a polysaccharide and the like), a polymer of a hydrophilic amino acid (e.g., polylysine, polyaspartate and the like), a polyalkane oxide and polyvinyl pyrrolidone), a fatty acid group (e.g., a mono-carboxylic acid or a di-carboxylic acid), a fatty acid ester group, a lipid group (e.g., diacylglycerol group, sphingolipid group (e.g., ceramidyl)) or a phospholipid group (e.g., phosphatidyl ethanolamine group). Preferably, the organic moiety is bound to a predetermined site where the organic moiety does not impair the function (e.g., decrease the antigen binding affinity) of the resulting immunoconjugate compared to the non-conjugated antibody moiety. The organic moiety can have a molecular weight of about 500 Da to about 50,000 Da, preferably about 2000, 5000, 10,000 or 20,000 Da. Examples and methods for modifying polypeptides, e.g., antibodies, with organic moieties can be found, for example, in U.S. Pat. Nos. 4,179,337 and 5,612,460, PCT Publication Nos. WO 95/06058 and WO 00/26256, and U.S. Patent Application Publication No. 20030026805.

### Chimeric Antigen Receptors

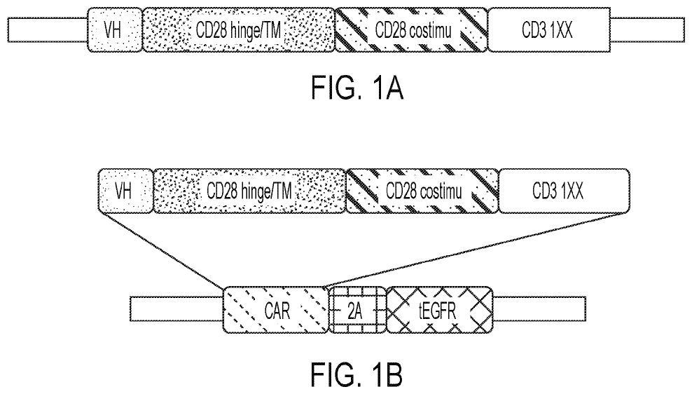

Chimeric Antigen Receptors (CARs) are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4):e23621). In the various embodiments of the GCC-specific CARs disclosed herein, the general scheme is set forth in FIG. 1. In some embodiments, the anti-GCC CARs comprise from the N-terminus to the C-terminus, a signal or leader peptide, an antigen binding domain, a transmembrane and/or hinge domain, a costimulatory domain, and an intracellular domain.

The present invention provides a CAR (e.g., a CAR polypeptide) that comprises an anti-GCC binding domain (e.g., a GCC binding domain as described herein), a transmembrane domain, and an intracellular signaling domain, and wherein said anti-GCC binding domain comprises a heavy chain complementary determining region 1 (HC CDR1), a heavy chain complementary determining region 2 (HC CDR2), and a heavy chain complementary determining region 3 (HC CDR3) of any anti-GCC heavy chain binding domain amino acid sequences listed in Table 1 or 8. In some embodiments, the anti-GCC CARs comprise from the N-terminus to the C-terminus, a signal or leader peptide, anti-GCC VH, CD28 transmembrane and hinge, CD28 costimulatory domain, and CD3 zeta intracellular domain.

The antigen binding domain can be any protein that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some embodiments, the antigen binding domain

The antigen-binding motif of a CAR is commonly fashioned after a single chain Fragment variable (ScFv), the minimal binding domain of an immunoglobulin (Ig) molecule or single domain antibody (For example, WO2018/028647A1). Alternate antigen-binding motifs, such as receptor ligands (i.e., IL-13 has been engineered to bind tumor expressed IL-13 receptor), intact immune receptors, library-derived peptides, and innate immune system effector molecules (such as NKG2D) also have been engineered.

The linking motifs of a CAR can be a relatively stable structural domain, such as the constant domain of IgG, or designed to be an extended flexible linker. In some embodiments, the anti-GCC binding domain (e.g., a polypeptide comprising a sequence provided in Table 1 or Table 8), is attached to the transmembrane domain via a linker, e.g., a linker described herein. In some embodiments, the anti-GCC CAR includes a (Gly4-Ser)n linker, wherein n is 1, 2, 3, 4, 5, or 6 (SEQ ID NO: 36).

Structural motifs, such as those derived from IgG constant domains, can be used to extend the ScFv binding domain away from the T-cell plasma membrane surface. This may be important for some tumor targets where the binding domain is particularly close to the tumor cell surface membrane (such as for the disialoganglioside GD2; Orentas et al., unpublished observations). To date, the signaling motifs used in CARs always include the CD3-ζ chain because this core motif is the key signal for T cell activation. The first reported second-generation CARs featured CD28 signaling domains and the CD28 transmembrane sequence. This motif was used in third-generation CARs containing CD137 (4-1BB) signaling motifs as well (Zhao Y et al. J Immunol. 2009; 183 (9): 5563-74). With the advent of new technology, the activation of T cells with beads linked to anti-CD3 and anti-CD28 antibody, and the presence of the canonical “signal 2” from CD28 was no longer required to be encoded by the CAR itself. Using bead activation, third-generation vectors were found to be not superior to second-generation vectors in in vitro assays, and they provided no clear benefit over second-generation vectors in mouse models of leukemia (Haso W, Lee D W, Shah N N, Stetler-Stevenson M, Yuan C M, Pastan I H, Dimitrov D S, Morgan R A, FitzGerald D J, Barrett D M, Wayne A S, Mackall C L, Orentas R J. Anti-CD22-chimeric antigen receptors targeting B cell precursor acute lymphoblastic leukemia, Blood. 2013; 121 (7):1165-74; Kochenderfer J N et al. Blood. 2012; 119 (12):2709-20). This is borne out by the clinical success of CD19-specific CARs that are in a second generation CD28/CD3-ζ (Lee D W et al. American Society of Hematology Annual Meeting. New Orleans, La.; Dec. 7-10, 2013) and a CD137/CD3-ζ signaling format (Porter D L et al. N Engl J Med. 2011; 365 (8): 725-33). In addition to CD137, other tumor necrosis factor receptor superfamily members such as OX40 also are able to provide important persistence signals in CAR-transduced T cells (Yvon E et al. Clin Cancer Res. 2009; 15(18):5852-60). Equally important are the culture conditions under which the CAR T-cell populations were cultured.

### Transmembrane Domain

With respect to the transmembrane domain, in various embodiments, a CAR can be designed to comprise a transmembrane domain that is attached to the extracellular domain of the CAR (e.g., the anti-GCC antigen binding domain. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region).

In one aspect, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the CAR-expressing cell, e.g., CART cell, surface. In a different aspect the amino acid sequence of the transmembrane domain may be modified or substituted so as to minimize interactions with the binding domains of the native binding partner present in the same CAR-expressing cell, e.g., CART.

As described herein, the CAR comprises a transmembrane domain. With respect to the transmembrane domain, the CAR comprises one or more transmembrane domains fused to the extracellular GCC antigen binding domain of the CAR. The transmembrane domain may be derived either from a natural or from a synthetic source. Where the source is natural, the domain may be derived from any membrane-bound or transmembrane protein.

Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. In some embodiments, the linker is a glycine-serine doublet or a triple alanine linker.

In some embodiments, the transmembrane domain that naturally is associated with one of the domains in the CAR is used in addition to the transmembrane domains described supra. In some embodiments, the transmembrane domain can be selected by amino acid substitution to avoid binding of such domains to the transmembrane domains of the same or different surface membrane proteins to minimize interactions with other members of the receptor complex.

### Intracellular Domain

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

### Substitutions and Variants

In some embodiments, amino acid sequence variants of the antibodies provided herein are contemplated. For example, it may be desirable to improve the binding affinity and/or other biological properties of the antibody or antibody fragment, e.g. sdAb. Amino acid sequence variants of an antibody may be prepared by introducing appropriate modifications into the nucleic acid sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In some embodiments, antibody variants having one or more amino acid substitutions are provided. Sites of interest for substitutional mutagenesis include the HVRs and FRs. As further described below in reference to amino acid side chain classes. Amino acid substitutions may be introduced into an antibody of interest and the products screened for a desired activity, e.g., retained/improved antigen binding, decreased immunogenicity, or improved ADCC or CDC.

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

In some aspects, the antigen binding domain is humanized. A non-human antibody is humanized, where specific sequences or regions of the antibody are modified to increase similarity to an antibody naturally produced in a human or fragment thereof. In one aspect, the antigen binding domain is humanized. A humanized antibody (or antigen binding fragment) can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions, e.g., conservative substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well known in the art.

One type of substitutional variant involves substituting one or more hypervariable region residues of a parent antibody (e.g., a humanized or human antibody). Generally, the resulting variant (s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots”, i.e., residues encoded by codons that undergo mutation at high frequency during the somatic maturation process (see, e.g., Chowdhury, Methods Mol. Biol. 207: 179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant VH sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15: 26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present application may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about f 3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336: 1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249: 533-545 (1986); US Patent Application No. US 2003/0157108 A1, Presta, L; and WO 2004/056312 A1, Adams et al.,), and knockout cell lines, such as alpha-1, 6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94 (4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

The CARs (including functional portions and functional variants thereof) can be obtained by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the CARs (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARs can be synthetic, recombinant, isolated, and/or purified.

### Detectable Markers and Tags

An antibody antigen binding fragments thereof, specific for one or more of the antigens disclosed herein, can also expressed with (e.g., co-expressed) with a tag protein. In some embodiments, a furin recognition site and downstream 2A self-cleaving peptide sequence, designed for simultaneous bicistronic expression of the tag sequence and the antibody sequence. In some embodiments, the 2A sequence comprises the nucleic acid sequence of GSGATNFSLLKQAGDVEENPGP SEQ ID NO. 3. In some embodiments, furin and P2A sequence comprises the nucleic acid sequence that encodes the amino acid sequence of SEQ ID NO: 3. In some embodiments, the P2A tag comprises the amino acid sequence of SEQ ID NO: 3 or a sequence with at least 95%, at least 96%, at least 97%, at least 98%, at least 99% identity thereof.

In some embodiments, an antibody or antigen binding fragments thereof, specific for one or more of the antigens disclosed herein, can also expressed with EGFR. In some embodiments, the antibody or antigen binding fragments thereof, specific for one or more of the antigens disclosed herein, are expressed with (e.g., co-expressed) truncated EGFR (tEGFR). In some embodiments, tEGFR comprises an amino acid sequence that is at least 95% identical, at least 96% identical, at least 97% identical, at least 98% identical, at least 99% identical or 100% identical to SEQ ID NO: 35.

tEGFR:
(SEQ ID NO: 35)
MLLLVTSLLLCELPHPAFLLIPRKVCNGIGIGEFKDSLSIN
ATNIKHFKNCTSISGDLHILPVAFRGDSFTHTPPLDPQEL
DILKTVKEITGFLLIQAWPENRTDLHAFENLEIIRGRTKQ
HGQFSLAVVSLNITSLGLRSLKEISDGDVIISGNKNLCYA
NTINWKKLFGTSGQKTKIISNRGENSCKATGQVCHALCSP
EGCWGPEPRDCVSCRNVSRGRECVDKCNLLEGEPREFVEN
SECIQCHPECLPQAMNITCTGRGPDNCIQCAHYIDGPHCV
KTCPAGVMGENNTLVWKYADAGHVCHLCHPNCTYGCTGPG
LEGCPTNGPKIPSIATGMVGALLLLLVVALGIGLFM
M

An antibody or antigen binding fragments thereof, specific for one or more of the antigens disclosed herein, can also be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRT), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP).

An antibody or antigen binding portion thereof, can also be conjugated with enzymes that are useful for detection, such as horseradish peroxidase, β-galactosidase, luciferase, alkaline phosphatase, glucose oxidase and the like. When an antibody, or antigen binding portion thereof, is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody, or antigen binding portion thereof, may also be conjugated with biotin, and detected through indirect measurement of avidin or streptavidin binding. It should be noted that the avidin itself can be conjugated with an enzyme or a fluorescent label.

An antibody, or antigen binding portion thereof, may be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

An antibody, or antigen binding portion thereof, can also be conjugated with a radiolabeled amino acid. The radiolabel may be used for both diagnostic and therapeutic purposes. For instance, the radiolabel may be used to detect one or more of the antigens disclosed herein and antigen expressing cells by x-ray, emission spectra, or other diagnostic techniques. Further, the radiolabel may be used therapeutically as a toxin for treatment of tumors in a subject, for example for treatment of a neuroblastoma. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Nucleic Acids, Expression Vectors, and Host Cells

Further provided by an embodiment of the invention is a nucleic acid comprising a nucleotide sequence encoding an antibody, or antigen binding portion thereof, described herein (including functional portions and functional variants thereof). The nucleic acids of the invention may comprise a nucleotide sequence encoding any of the leader sequences, antigen binding domains, transmembrane domains, and/or intracellular T cell signaling domains described herein. In one aspect, the present invention encompasses a recombinant nucleic acid construct comprising a nucleic acid molecule encoding an antibody or fragment thereof, wherein the nucleic acid molecule comprises the nucleic acid sequence encoding an anti-GCC binding domain. In one aspect, the present invention provides a nucleic acid encoding a VH amino acid sequence according to SEQ ID Nos: 1, 20, 21, 26, 27, or 28 or a sequence having at least 75%, 80%, 90% or 95% sequence identity to SEQ ID Nos: 1, 20, 21, 26, 27, or 28. In some embodiments, the nucleic acid comprises a sequence that is at least 75%, 80%, 90%, 95%, 96%, 97%, 98%, 99% or 100% identical to SEQ ID Nos: 30-34.

In some embodiments, the nucleotide sequence may be codon-modified. Without being bound to a particular theory, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In one aspect aspect, the invention pertains to a vector comprising a nucleic acid molecule described herein, e.g., a nucleic acid molecule encoding an antibody or antigen binding fragment described herein. In one embodiment, the vector is selected from the group consisting of a DNA, a RNA, a plasmid, a lentiviral vector, adenoviral vector, or a retrovirus vector.

In one embodiment, the vector is a lentiviral vector. In one embodiment, the vector further comprises a promoter. In one embodiment, the promoter is an EF-1 promoter.

Expression vectors include plasmids, retroviruses, cosmids, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Suitable expression vectors can contain a number of components, for example, an origin of replication, a selectable marker gene, one or more expression control elements, such as a transcription control element (e.g., promoter, enhancer, or terminator) and/or one or more translation signals, a signal sequence or leader sequence, and the like. Polyadenylation and transcription termination occur at native chromosomal sites downstream of the coding regions. The resulting chimeric antibody may be joined to any strong promoter. Examples of suitable vectors that can be used include those that are suitable for mammalian hosts and based on viral replication systems, such as simian virus 40 (SV40), Rous sarcoma virus (RSV), adenovirus 2, bovine papilloma virus (BPV), papovavirus BK mutant (BKV), or mouse and human cytomegalovirus (CMV), and moloney murine leukemia virus (MMLV), native Ig promoters, etc. A variety of suitable vectors are known in the art, including vectors which are maintained in single copy or multiple copies, or which become integrated into the host cell chromosome, e.g., via LTRs, or via artificial chromosomes engineered with multiple integration sites (Lindenbaum et al. Nucleic Acid Res. 32:e172 (2004), Kennard et al. Biotechnol. Bioeng. Online May 20, 2009). Additional examples of suitable vectors are listed in a later section.

The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one embodiment, the template includes sequences for the CAR. In an embodiment, an RNA CAR vector is transduced into a cell, e.g., T cell or NK cell, by electroporation.

Thus, the invention provides an expression vector comprising a nucleic acid encoding an antibody, antigen-binding fragment of an antibody (e.g., a human, humanized, chimeric antibody or antigen-binding fragment of any of the foregoing), antibody chain (e.g., heavy chain, light chain) or antigen-binding portion of an antibody chain that binds a GCC protein.

Expression in eukaryotic host cells is useful because such cells are more likely than prokaryotic cells to assemble and secrete a properly folded and immunologically active antibody. However, any antibody produced that is inactive due to improper folding may be renaturable according to known methods (Kim and Baldwin, “Specific Intermediates in the Folding Reactions of Small Proteins and the Mechanism of Protein Folding”, Ann. Rev. Biochem. 51, pp. 459-89 (1982)). It is possible that the host cells will produce portions of intact antibodies, such as light chain dimers or heavy chain dimers, which also are antibody homologs according to the present invention.

In an embodiment, the nucleic acids can be incorporated into a recombinant expression vector. In this regard, an embodiment provides recombinant expression vectors comprising any of the nucleic acids. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally-occurring as a whole.

However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.).

Bacteriophage vectors, such as λ, λZapII (Stratagene), EMBL4, and λNMI 149, also can be used. Examples of plant expression vectors include pBIO1, pBI101.2, pBHO1.3, pBI121 and pBIN19 (Clontech). Examples of animal expression vectors include pEUK-C1, pMAM, and pMAMneo (Clontech). The recombinant expression vector may be a viral vector, e.g., a retroviral vector or a lentiviral vector. A lentiviral vector is a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include, for example, and not by way of limitation, the LENTIVECTOR® gene delivery technology from Oxford BioMedica plc, the LENTIMAX™ vector system from Lentigen and the like. Nonclinical types of lentiviral vectors are also available and would be known to one skilled in the art.

A number of transfection techniques are generally known in the art (see, e.g., Graham et al., Virology, 52: 456-467 (1973); Sambrook et al., supra; Davis et al., Basic Methods in Molecular Biology, Elsevier (1986); and Chu et al, Gene, 13: 97 (1981).

Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al, Nature, 327: 70-73 (1987)).

In an embodiment, the recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the antibody or antigen binding fragment thereof, or to the nucleotide sequence which is complementary to or which hybridizes to the nucleotide sequence encoding the antibody or antigen binding fragment thereof. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, EF1 alpha promoter or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

An embodiment further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, HEK293T cells, and the like. For purposes of amplifying or replicating the recombinant expression vector, the host cell may be a prokaryotic cell, e.g., a DH5a cell. For purposes of producing a recombinant antibody or antigen binding fragment thereof, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

One aspect of the present application provides an engineered immune effector cell, comprising any one of the antibodies or antigen binding fragments thereof described herein, or any one of the isolated nucleic acids described above, or any one of the vectors described above

Also provided by an embodiment is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques.

Alternatively, the gene of interest can be produced synthetically, rather than cloned. The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non proliferating cells, such as hepatocytes. They also have the added advantage of low immunogenicity.

The expression of natural or synthetic nucleic acids encoding an anti-GCC binding agent described herein may be achieved by operably linking a nucleic acid encoding the an anti-GCC binding agent polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466 incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. Further, the expression vector may be provided to a cell in the form of a viral vector.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant vims can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto.

Another example of a suitable promoter is Elongation Growth Factor-1a (EF-1a). However, other constitutive promoter sequences may also be used, including, but not limited to the simian vims 40 (SV40) early promoter, mouse mammary tumor virus (MMTV), human immunodeficiency vims (HIV) long terminal repeat (LTR) promoter, MoMuLV promoter, an avian leukemia vims promoter, an Epstein-Barr vims immediate early promoter, a Rous sarcoma vims promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of an anti-GCC binding agent (e.g., a single domain antibody) polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure.

Both selectable markers and reporter genes may be flanked with appropriate regulatory sequences to enable expression in the host cells. Useful selectable markers include, for example, antibiotic-resistance genes, such as neo and the like. Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex vims 1, adenoviruses and adeno-associated viruses, and the like. See, for example, U.S. Pat. Nos. 5,350,674 and 5,585,362. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes.

An exemplary colloidal system for use as a delivery vehicle in vitro and in vivo is a liposome (e.g., an artificial membrane vesicle). In the case where a non-viral delivery system is utilized, an exemplary delivery vehicle is a nanoparticle, e.g., a liposome or other suitable sub-micron sized delivery system. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al, 1991 Glycobiology 5: 505-10).

However, compositions that have different structures in solution than the normal vesicular structure are also encompassed. For example, the lipids may assume a micellar structure or merely exist as nonuniform aggregates of lipid molecules. Also contemplated are lipofectaminenucleic acid complexes. Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising an anti-GCC binding agent (e.g., a single domain antibody) encoding nucleic acid molecule. In one aspect, an anti-GCC binding agent (e.g., a single domain antibody) vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the anti-GCC binding agent construct in mammalian T cells. In one aspect, the mammalian T cell is a human T cell.

In some aspects, non-viral methods can be used to deliver a nucleic acid encoding a anti-GCC binding agent described herein into a cell or tissue or a subject. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome.

Additional and exemplary transposons and non-viral delivery methods are described on pages 196-198 of International Application WO 2016/164731, filed Apr. 8, 2016, which is incorporated by reference in its entirety.

### Methods of Treatment

The present invention relates methods of treatment comprising administering an anti-GCC antigen binding molecule as described herein to a subject. In some embodiments, anti-GCC antigen binding molecules (e.g., single domain antibody) disclosed herein can be used in methods of treating or preventing a disease in a mammal. In this regard, an embodiment provides a method of treating or preventing cancer in a mammal, comprising administering to the mammal the antigen binding molecules (e.g., single domain antibody), the nucleic acids, the recombinant expression vectors, the host cells, the population of cells, the antibodies and/or the antigen binding portions thereof, and/or the pharmaceutical compositions in an amount effective to treat or prevent cancer in the mammal. The invention also relates to an anti-GCC antigen binding molecule as described herein (e.g. a sdAb) for use in the treatment of a disease. The invention also relates to an anti-GCC antigen binding molecule as described herein (e.g. a sdAb) for use in the treatment of cancer. The invention also relates to an anti-GCC antigen binding molecule as described herein (e.g. a sdAb) for use in the manufacture of a medicament for the treatment of cancer.

The administration of the compositions described herein may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one embodiment, the compositions described herein, e.g., comprising an antigen binding molecules (e.g., single domain antibody)-expressing cell, are administered to a patient by intradermal or subcutaneous injection. In one embodiment, the the compositions described herein, e.g., comprising an antigen binding molecules (e.g., single domain antibody)-expressing cell, are administered by i.v. injection. The compositions described herein, e.g., comprising a antigen binding molecules (e.g., single domain antibody)-expressing cell, may be injected directly into a tumor, lymph node, or site of infection.

For purposes of the methods, wherein host cells or populations of cells are administered, the cells can be cells that are allogeneic or autologous to the mammal. Preferably, the cells are autologous to the mammal. As used herein, allogeneic means any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. As used herein, “autologous” means any material derived from the same individual to whom it is later to be re-introduced into the individual.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). In some embodiments, the mammal is a human.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., meduloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

In certain embodiments, the cancer is a gastrointestinal cancer. In some embodiments, the cancer is gastric cancer. In some embodiments, the cancer is colorectal cancer. In some embodiments, the cancer is colon cancer. In some embodiments, the cancer has abnormal expression of GCC.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of cancer in a mammal.

Furthermore, the treatment or prevention provided by the method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment provides a method of detecting the presence of cancer in a mammal, comprising: (a) contacting a sample comprising one or more cells from the mammal with the antigen binding molecules (e.g., single domain antibody) the antigen binding portions thereof, or the pharmaceutical compositions, thereby forming a complex, (b) and detecting the complex, wherein detection of the complex is indicative of the presence of cancer in the mammal. In some embodiments, the contacting can take place in vitro or in vivo with respect to the mammal. In some embodiments, the contacting is in vitro.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the method of detecting the presence of a proliferative disorder, e.g., cancer, in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

Also, detection of the complex can occur through any number of ways known in the art. For instance, the antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles) as disclosed supra.

Methods of testing antigen binding molecules (e.g., single domain antibody) for the ability to recognize target cells and for antigen specificity are known in the art. For instance, Clay et al., J. Immunol, 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)).

Another embodiment provides for the use of antigen binding molecules (e.g., single domain antibody or antigen binding portions thereof), and/or pharmaceutical compositions of the invention, for the treatment or prevention of a proliferative disorder, e.g., cancer, in a mammal. The cancer may be any of the cancers described herein.

Any method of administration can be used for the disclosed therapeutic agents, including local and systemic administration. For example topical, oral, intravascular such as intravenous, intramuscular, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used, for example, a chemotherapeutic agent may be administered orally and an antibody or antigen binding fragment or conjugate or composition may be administered intravenously. Methods of administration include injection for which the antibodies, antigen binding fragments, or compositions are provided in a nontoxic pharmaceutically acceptable carrier such as water, saline, Ringer's solution, dextrose solution, 5% human serum albumin, fixed oils, ethyl oleate, or liposomes. In some embodiments, local administration of the disclosed compounds can be used, for instance by applying the antibody or antigen binding fragment to a region of tissue from which a tumor has been removed, or a region suspected of being prone to tumor development. In some embodiments, sustained intra-tumoral (or near-tumoral) release of the pharmaceutical preparation that includes a therapeutically effective amount of the antibody or antigen binding fragment may be beneficial. In other examples, the conjugate is applied as an eye drop topically to the cornea, or intravitreally into the eye.

The disclosed therapeutic agents can be formulated in unit dosage form suitable for individual administration of precise dosages. In addition, the disclosed therapeutic agents may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

In one embodiment, the present disclosure provides pharmaceutical compositions comprising at least one therapeutic agent of the present disclosure (e.g., a therapeutic agent of the present disclosure) or a pharmaceutically acceptable salt thereof together with a pharmaceutically acceptable carrier suitable for administration to a human or animal subject, either alone or together with other anti-cancer agents.

In one embodiment, the present disclosure provides methods of treating human or animal subjects suffering from a cellular proliferative disease, such as cancer. The present disclosure provides methods of treating a human or animal subject in need of such treatment, comprising administering to the subject a therapeutically effective amount of a therapeutic agent of the present disclosure or a pharmaceutically acceptable salt thereof, either alone or in combination with other anti-cancer agents.

In particular, compositions will either be formulated together as a combination therapeutic or administered separately. In combination therapy, the compound of the present disclosure and other anti-cancer agent(s) may be administered either simultaneously, concurrently or sequentially with no specific time limits, wherein such administration provides therapeutically effective levels of the two compounds in the body of the patient.

In some embodiments, the compound of the present disclosure and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination.

A compound of the present disclosure may in particular be used as a radiosensitizer, especially for the treatment of tumors which exhibit poor sensitivity to radiotherapy.

In another aspect, the invention provides a kit, e.g. for the treatment or prevention of a disease or an immune response and/or for detecting GCC for diagnosis, prognosis or monitoring disease comprising an antibody, e.g. single domain antibody as described herein. Such a kit may contain other components, packaging, instructions, or material to aid in the detection of GCC protein. The kit may include a labeled single domain antibody or binding agent as described herein and one or more compounds for detecting the label.

Unless stated otherwise, all technical and scientific terms and phrases used herein have the same meaning as commonly understood by one of ordinary skill in the art. Although any methods and materials similar or equivalent to those described herein can be used in the practice or testing of the present invention, the preferred methods and materials are now described. All publications mentioned herein are incorporated herein by reference.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

All publications, patent applications, patents and other references mentioned herein are incorporated by reference in their entirety. The present invention will be more fully understood by reference to the following Examples.

### EXAMPLES

These Examples are set forth to aid in the understanding of the invention but are not intended to, and should not be construed to limit its scope in any way. The Examples do not include detailed descriptions of conventional methods that would be well known to those of ordinary skill in the art (molecular cloning techniques, etc.).

### Example 1. Isolation and Selection of GCC V_{H} Antibodies

Single domain heavy chain antibodies (V_{H}) were generated that specifically target human GCC (e.g., anti-GCC binders provided in Tables 1-3). As shown in Table 5, recombinant single domain anti-GCC V_{H} constructs were assessed for binding affinity to GCC in vitro. Binding kinetics were determined using an Octet binding assay. These were measured in real-time bio-layer interferometer based biosensor Octet (ForteBio). All the binding studies were performed in HBS-ET Octet kinetics buffer. Biosensors were always washed in Octet kinetics buffer in between different steps. A seven point, two-fold dilution series of each V_{H} was made. The contact time for each of the association steps was 300 seconds and the dissociation step was varied between 400-600 seconds. Kinetic association (ka) and dissociation (kd) rate constants were determined by processing and fitting the data to a 1:1 binding model using ForteBio Analysis software. The calculated affinity and kinetic constants are shown in Table 4.

Binding of single domain antibodies to CT26 cells was measured using flow cytometry. CT26 cells were incubated with serially diluted V_{H} and fluorescence was measured by flow cytometry. Anti-GCC V_{H} antibodies were confirmed for binding to CT26 cells by a FACS dose response assay to obtain an on cell binding EC50 (nM) as shown in Table 4.

TABLE 4
Binding Affinity of GCC VH antibodies

| GCC | | | |
|---|---|---|---|
| Antigen | | | on cell |
| Binding | | | binding EC50 |
| agent | Koff (1/s) | KD(M) | (M) |
| V1 | 0.00020795 | 3.2935E−10 | 7.2E−10 |
| V5 | 0.000718667 | 7.713E−09 | 1.52E−09 |
| V36 | 0.0035 | 0.000000105 | N/A |
| V48 | 0.000341 | 1.375E−09 | 1.01E−09 |
| V51 | 0.00185 | 9.544E−09 | N/A |

To determine stability, V_{H} constructs were subjected to size exclusion chromatography (SEC). Briefly, purified V_{H} were stored at varied concentrations in PBS buffer overnight at 4° C., and then analysed at various time points using a SEC column. Samples were injected in a sodium phosphate buffer. Data were collected over time and the area of monomer peak remaining after storage as compared to that present at the start (T=0) was calculated. Stability results were obtained as shown in Table 5 below.

TABLE 5
Overnight stability using SEC

| Anti-GCC | % 4° C. | | |
|---|---|---|---|
| VH antibody | Monomer (%) | Purity (%) | RT (min) |
| V1 | 108.4 | 91.5 | 3.586 |
| V5 | 123.04 | 79.2 | 3.17 |
| V36 | 92.43 | 79.36 | 3.08 |
| V48 | 104.54 | 73.91 | 3.208 |
| V51 | 109.2 | 59.22 | 3.304 |

ELISA were performed to measure VH binding to human light chains. Binding values for each of the VH (measured as OD450 nm) were less than 0.1, which is the background signal for this assay.

### Example 2. Design and Characterization of GCC Chimeric Antigen Receptors

Chimeric antigen receptor constructs are engineered to include an extracellular binding domain (e.g., anti-GCC binder sequence) comprising the single domain antibodies described above (e.g., a VH provided in Table 2 or Table 3). CAR T constructs are generated by linking the binder sequence in frame to CD28 hinge/transmembrane domains and costimulatory domain and CD3 zeta-lxx signaling domain. Schematics of exemplary CAR constructs are shown in FIGS. 1A and 1B.

Nucleic acids encoding the CAR construct sequences were cloned into a retroviral plasmid backbone. Retroviral vector containing supernatants were generated by transient transfection of phenix ampho cells (ATCC CRL-3213) and retroviral vector-containing supernatants were harvested, and stored at −80° C.

Human primary T cells from healthy donors were purified from Peripheral blood mononuclear cell (PBMC) isolated from leukopaks (purchased from commercial provider with donor's written consent) using immunomagnetic bead selection of CD3+ cells according to manufacturer's protocol (EasySep™ Human T Cell Isolation Kit, Stem Cell Technologies #17951). T cells were cultured in X-vivo 15 medium (Lonza #04-744Q) supplemented with 10% Penicillin-Streptomycin (Gibco 15140-122) and 2 ng/ml IL-2 (Milteyni 130-097-743) at a density of 1 million cells/ml. Cells were activated with CD3/CD28 MACS® T Cell TransAct reagent (Miltenyi Biotec MACS #130-111-160) and transduced on day 2 or day 3 with retroviral vectors encoding CAR constructs overnight. The next day, CAR-T cell cultures were transferred to a G-Rex6® Well Plate (WilsonWolf P/N 80240M) and propagated in X-vivo 15 medium (Lonza #04-744Q) supplemented with 10% Penicillin-Streptomycin (Gibco 15140-122) and 2 ng/ml IL-2 (Milteyni 130-097-743) until harvest on day 7-10. Medium change and IL-2 replenishment were performed every 2-3 days.

CAR T cell expression was evaluated by flow cytometry using an anti-EGFR antibody (R&D systems: FAB9577R) or soluble GCC extracellar domain recombinant protein for CAR surface expression.

### Example 3. GCC CAR T Cell Activity In Vitro

The present Example describes anti-GCC CAR T cell activity in vitro. CAR-T cell cytotoxicity against GCC-expressing and GCC-negative target cancer cell lines was examined. The target cancer cell lines included GSU, LS1034 and HT55, which endogenously express GCC, as well as HT29-GCC, a human colorectal cancer cell line engineered to stably express GCC, and its vector control cell line that is GCC-negative, HT29-vec. Each target cell line was seeded in a 384-well plate, and GCC CAR-T or non-targeting CAR-T cells (negative control) were added at effector-to-target (E:T) ratios of 10:1, 3:1, 1:1 and 0.3:1. Wells with target cells only and wells with effector cells only were included as controls. After two days, cell viability was measured using CellTiter-Glo® One Solution Assay (Promega, G8462). Percent viability of target cells was calculated from the luminescence signals of the co-culture wells, after first subtracting the signals of the effector-cells-only wells, then dividing by the signals of the target-cell-only wells. Percent killing was calculated by subtracting the percent viability of target cells from 100%. GCC CAR-T cells.

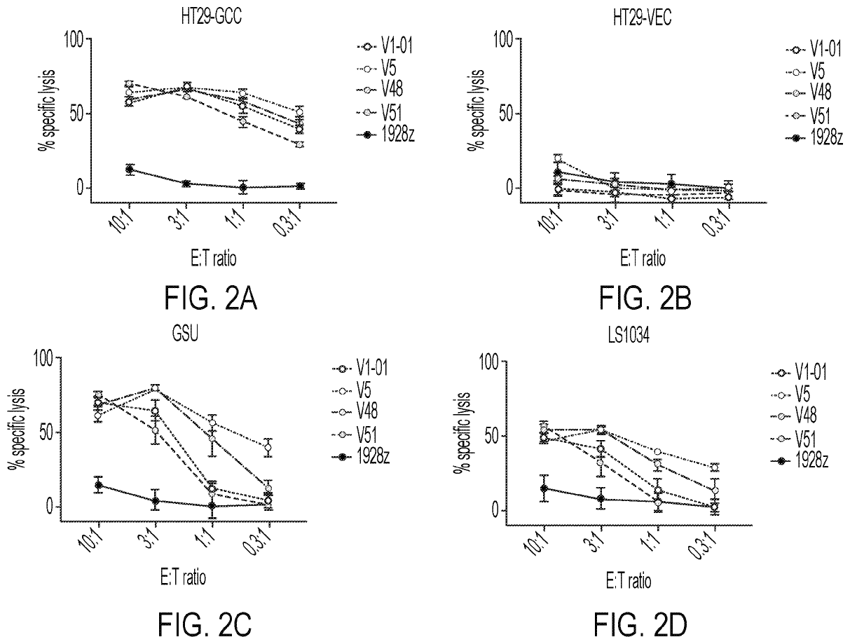

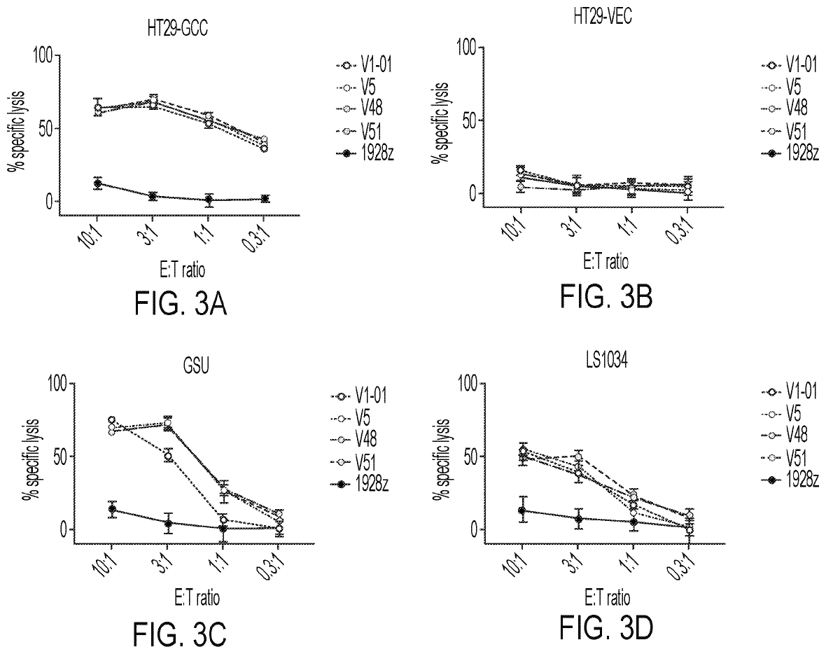

V_{H} anti-GCC binders exhibited cell killing against GCC-expressing target cell lines, in contrast to non-targeting CD19 CAR-T cells (1928z-lxx) used as a control. As shown in FIG. 2A-2D, CAR-T cells expressing anti-GCC CARs in the absence of truncated EGFR (tEGFR) demonstrated in vitro cytotoxicity against GCC expressing cells HT29-GCC cells, human colorectal cancer cell line HT29 engineered to stably express GCC) (FIG. 2A); and endogenously expressing GCC cell lines GSU (FIG. 2C) and LS1034 (FIG. 2D). As shown in FIG. 3A-3D, CAR-T cells expressing anti-GCC CARs in the presence of truncated EGFR (tEGFR) also demonstrated in vitro cytotoxicity against GCC expressing cells HT29-GCC (FIG. 3A); GSU (FIG. 3C) and LS1034 (FIG. 3D). Bars represent mean+SD values from three technical replicates. Data are representative of >3 independent experiments performed with anti-GCC CAR T cells from >3 donors. GCC CAR-T cells did not exhibit cell killing against the GCC-negative HT29-vec cells (FIG. 2B and FIG. 3B), indicating GCC CAR-T cell killing activity was antigen-dependent.

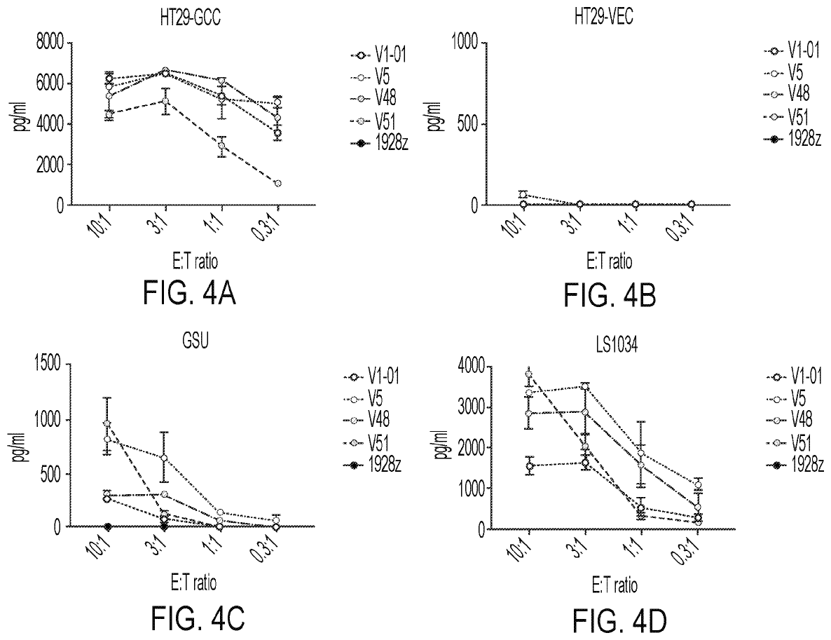

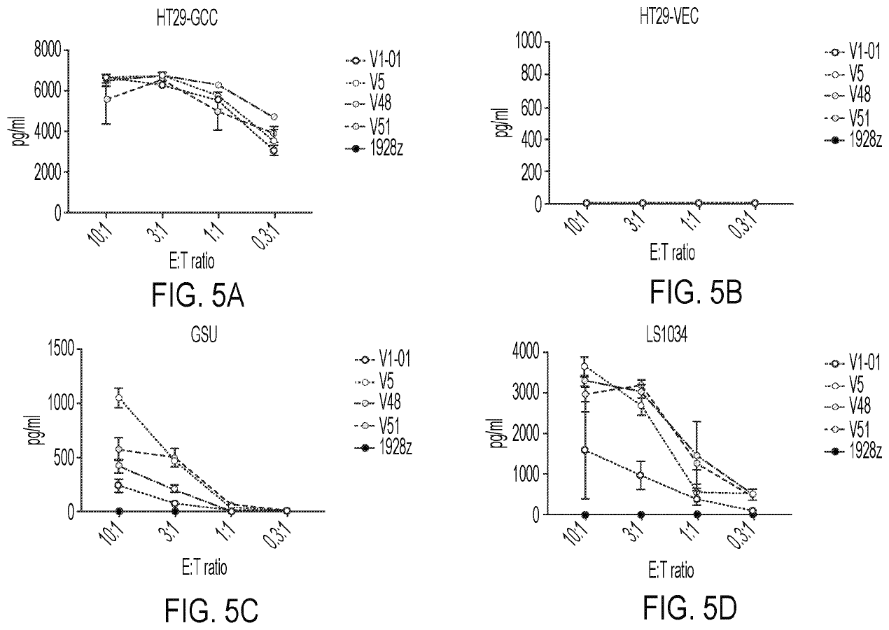

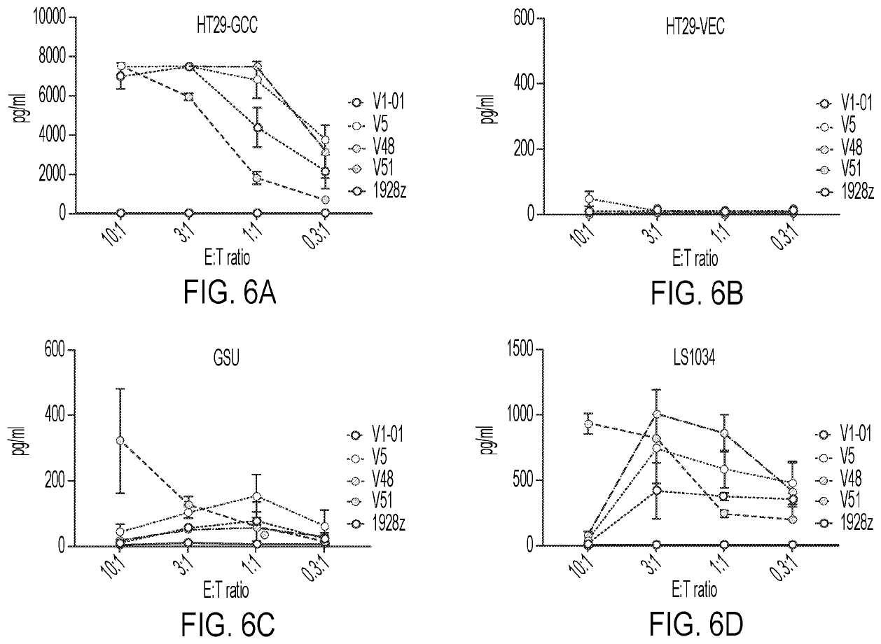

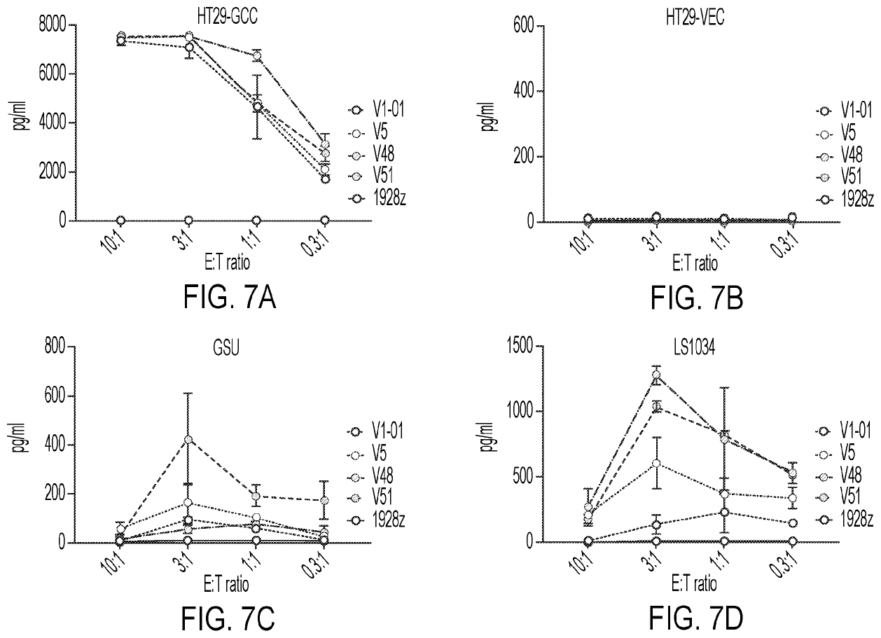

In addition to antigen-dependent cell killing, the in vitro activity of GCC CAR-T cells was also assessed by evaluating its antigen-dependent secretion of IFNγ and IL2. GCC CAR-T cells with anti-GCC V_{H} binders were co-cultured with GCC-expressing and GCC-negative target cancer cell lines at E:T ratios of 10:1, 3:1, 1:1 and 0.3:1. Supernatant was collected after two days of co-culture. Secreted IFNγ and IL2 in the supernatant were detected using the Intellicyt QBeads Human PlexScreen kit (Sartorius, 90702). GCC CAR-T cells with all VH binders secreted IFNγ both in the presence of tEGFR (5A-5D) and the absence of (4A-4D) when co-cultured with GCC-expressing target cells, but not when co-cultured with GCC-negative target cells (FIGS. 4B and 5B), indicating antigen-dependent cytokine release. GCC CAR-T cells with all V_{H} binders secreted IL2 both in the presence (7A-7D) and absence of tEGFR (6A-6D) when co-cultured with GCC-expressing target cells, but not when co-cultured with GCC-negative target cells (FIGS. 6B and 7B), indicating antigen-dependent cytokine release.

Having thus described several aspects of at least one embodiment of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

### Table of Sequences

Table 6 below provides descriptions and sequences disclosed herein.

TABLE 6
Table of Sequences

| SEQ | | |
|---|---|---|
| ID | | |
| NO | Description | Sequence |
| 1 | V1 VH | QVQLQESGPGLVKPSETLSL |
| | | TCTVSGASISHYYWSWFRQP |
| | | AGKGLEWIGRIYPSGSTSYN |
| | | PSLKSRVAMSVDTPKNQFSL |
| | | NLSSVTAADTAVYYCARDRS |
| | | TGWSEWNSDLWGRGTLVTVS |
| | | S |
| 2 | v31 leader | MEFGLSWVFLVAIIKGVQC |
| 3 | P2A | GSGATNFSLLKQAGDVEENP |
| | | GP |
| 4 | Leader | MALPVTALLLPLALLLHAAR |
| | sequence | P |
| 5 | GCC AA | MKTLLLDLALWSLLFQPGWL |
| | Sequence | SFSSQVSQNCHNGSYEISVL |
| | | MMGNS |
| | | AFAEPLKNLEDAVNEGLEIV |
| | | RGRLQNAGLNVTVNATFMYS |
| | | DGLIHNSGDCRSSTCEGLDL |
| | | LRKISNAQRMGCVLIGPSCT |
| | | YSTFQMYLDTELSYPMISAG |
| | | SFGLSCDYKETLTRLMSPAR |
| | | KLMYFLVNFWKTNDLPFKTY |
| | | SWSTSYVYKNGTETEDCFWY |
| | | LNALEASVSYFSHELGFKVV |
| | | LRQDKEFQDILMDHNRKSNV |
| | | IIMCGGPEFLYKLKGDRAVA |
| | | EDIVIILVDLENDQYFEDNV |
| | | TAPDYMKNVLVLTLSPGNSL |
| | | LNSSFSRNLSPTKRDFALAY |
| | | LNGILLFGHMLKIFLENGEN |
| | | ITTPKFAHAFRNLTFEGYDG |
| | | PVTLDDWGDVDSTMVLLYTS |
| | | VDTKKYKVLLTYDTHVNKTY |
| | | PVDMSPTFTWKNSKLPNDIT |
| | | GRGPQILMIAVFTLTGAVVL |
| | | LLLVALLMLRKYRKDYELRQ |
| | | KKWSHIPPENIFPLETNETN |
| | | HVSLKIDDDKRRDTIQRLRQ |
| | | CKYDKKRVILKDLKHNDGNF |
| | | TEKQKIELNKLLQIDYYNLT |
| | | KFYGTVKLDTMIFGVIEYCE |
| | | RGSLREVLNDTISYPDGTFM |
| | | DWEFKISVLYDIAKGMSYLH |
| | | SSKTEVHGRLKSTNCVVDSR |
| | | MVVKITDFGCNSILPPKKDL |
| | | WTAPEHLRQANISQKGDVYS |
| | | YGIIAQEIILRKETFYTLSC |
| | | RDRNEKIFRVENSNGMKPFR |
| | | PDLFLETAEEKELEVYLLVK |
| | | NCWEEDPEKRPDFKKIETTL |
| | | AKIFGLFHDQKNESYMDTLI |
| | | RRLQLYSRNLEHLVEERTQL |
| | | YKAERDRADRLNFMLLPRLV |
| | | VKSLKEKGFVEPELYEEVTI |
| | | YFSDIVGFTTICKYSTPMEV |
| | | VDMLNDIYKSFDHIVDHHDV |
| | | YKVETIGDAYMVASGLPKRN |
| | | GNRHAIDIAKMALEILSFMG |
| | | TFELEHLPGLPIWIRIGVHS |
| | | GPCAAGVVGIKMPRYCLFGD |
| | | TVNTASRMESTGLPLRIHVS |
| | | GSTIAILKRTECQFLYEVRG |
| | | ETYLKGRGNETTYWLTGMKD |
| | | QKFNLPTPPTVENQQRLQAE |
| | | FSDMIANSLQKRQAAGIRSQ |
| | | KPRRVASYKKGTLEYLQLNT |
| | | TDKESTYF |
| 6 | Leader | MKHLWFFLLLVAAPRWVLS |
| | Sequence (v1) | |
| 7 | Leader | MELGLSWVFLVAILEGVQC |
| | Sequence (v5, | |
| | v36, v48, v51) | |
| 8 | V1 HCDR1 | HYYWS |
| 9 | V5 HCDR1 | RYWMS |
| 10 | V36 HCDR1 | RYWMT |
| 10 | V48 HCDR1 | RYWMT |
| 10 | V51 HCDR1 | RYWMT |
| 11 | V1 HCDR2 | RIYPSGSTSYNPSLKS |
| 12 | V5 HCDR2 | KIRHDGGEKYYVDSVKG |
| 13 | V36 HCDR2 | KIKYDGSEKYYADSVKG |
| 14 | V48 HCDR2 | KIRHDGGEKYYPDSVKG |
| 15 | V51 HCDR2 | KIRHDGGEKYYADSVKG |
| 16 | V1 HCDR3 | DRSTGWSEWNSDL |
| 17 | V5 HCDR3 | DYTRDV |
| 18 | V36 HCDR3 | DYNKDY |
| 19 | V48 HCDR3 | DYNKDL |
| 18 | V51 HCDR3 | DYNKDY |
| 20 | V1-01 | EVQLQESGPGLVKPSETLSL |
| | | TCTVSGASISHYYWSWFRQP |
| | | AGKGLEWIGRIYPSGSTSYN |
| | | PSLKSRVAMSVDTPKNQFSL |
| | | KLSSVTAADTAVYYCARDRS |
| | | TGWSEWNSDLWGRGTLVTVS |
| | | S |
| 21 | V5 | QVQLVESGGGLVQPGGSLRL |
| | | SCTASGFTFSRYWMSWVRQA |
| | | PGKGLEWVAKIRHDGGEKYY |
| | | VDSVKGRFTISRDNAKNSLY |
| | | LQMNSLRAEDTAVYYCATDY |
| | | TRDVWGQGTAVTVSS |
| 22 | V8 | QVQLVESGGGLVQPGGSLRL |
| | | SCAASGFTFSRYWMSWVRQA |
| | | PGKGLEWVAKIKYDGSEKYY |
| | | VDSVKGRFTISRDNAKNSVY |
| | | LQMNSLRAEDTGVYYCATDF |
| | | TRDVWGQGTTVTVSS |
| 23 | V9 | EVQLVESGGGLVQPGGSLRL |
| | | SCAASGFTFSRYWMTWVRQA |
| | | PGRGLEWVAKIRYDGGEKYY |
| | | VDSVKGRFTISRDNAKNSLY |
| | | LQMNSLRAEDTAVYYCATDF |
| | | TRDVWGQGTTVTVSS |
| 24 | V30 | QVQLVESGGGLVQPGGSLRL |
| | | SCAASGFNFGRYWMSWVRQA |
| | | PGKGREWVAKIKYDGSEKYY |
| | | VDSVKGRFTISRDNAKNSLY |
| | | LQMNSLRAEDTAVYYCATDF |
| | | TRDVWGQGTTVTVSS |
| 25 | V31 | QVQLVESGGGVVRPGGSLRL |
| | | SCAASGFTFSRYWMSWVRQA |
| | | PGKGREWVAKIKYDGSEKYY |
| | | ADSVKGRFTISRDNAKNSLY |
| | | LQMNSLRADDTAVYYCATDF |
| | | TRDVWGQGTTVTVSS |
| 26 | V36 | EVQLVESGGGLAQPGGSLRL |
| | | SCAASGFTFSRYWMTWVRQA |
| | | PGGRLEWVAKIKYDGSEKYY |
| | | ADSVKGRFTISRDNAKNSLY |
| | | LQMDSLRAEDTAVYYCTRDY |
| | | NKDYWGQGTLVTVSS |
| 27 | V48 | EVQLVESGGGLVQPGGSLRL |
| | | TCAASGFTFSRYWMTWVRQA |
| | | PGKGLEWVAKIRHDGGEKYY |
| | | PDSVKGRFTVSRDNAKNSLY |
| | | LQMDNLRAEDTAMYYCTRDY |
| | | NKDLWGQGTLVTVSS |
| 28 | V51 | EVQLVESGGGLVQPGGSLRL |
| | | SCAASGFTFSRYWMTWVRQA |
| | | PGKGLEWVAKIRHDGGEKYY |
| | | ADSVKGRFTISRDNAKNSLY |
| | | LQMNSLRAEDTAVYYCTRDY |
| | | NKDYWGQGTLVTVSS |
| 29 | heat-stable | NTFYCCELCCNPACAGCY |
| | enterotoxin | |
| 30 | V1-01 | GAGGTGCAGCTGGTGGAGTC |
| | Nucleic acid | TGGGGGAGGCTTGGTCCAGC |
| | sequence | CGGGGGGGTCCCTGAGACTC |
| | | TCCTGTGCAGCCTCTGGATT |
| | | CACCTTTAATAGTTATTGGA |
| | | TGAGTTGGGACCGCCAGGCT |
| | | CCAGGGAAGGGCCTGGAGTG |
| | | GGTGGCCAACATAAACCAAG |
| | | ATGGAAGTGAGAAATACTAT |
| | | GGGGACTCTGTGAGGGGCCG |
| | | ATTCACCATCTCCAGAGACA |
| | | ACGCCAAGAACACAGTGTAT |
| | | CTGCAAATGAACAGCCTGAG |
| | | AGCCGAGGACACGGCTGTGT |
| | | ATTACTGTGTGAGAAGGAGC |
| | | CACGGCGTCCGGGGGCAAGG |
| | | GACCACGGTCACCGTCTCCT |
| | | CA |
| 31 | V5 | CAGGTGCAGCTGGTGGAGTC |
| | Nucleic acid | TGGGGGAGGCTTGGTACAGC |
| | sequence | CTGGGGGGTCCCTGAGACTC |
| | | TCCTGTACAGCCTCTGGATT |
| | | CACCTTTAGTCGGTATTGGA |
| | | TGAGCTGGGTCCGCCAGGCT |
| | | CCAGGGAAGGGGCTGGAGTG |
| | | GGTGGCCAAGATAAGGCACG |
| | | ATGGAGGTGAGAAATACTAT |
| | | GTGGACTCTGTGAAGGGCCG |
| | | ATTCACCATCTCCAGAGACA |
| | | ACGCCAAGAATTCACTGTAT |
| | | CTGCAAATGAACAGCCTGAG |
| | | AGCCGAGGACACGGCTGTGT |
| | | ATTACTGTGCGACAGACTAT |
| | | ACGAGGGACGTCTGGGGCCA |
| | | AGGGACCGCGGTCACCGTCT |
| | | CCTCA |
| 32 | V36 | GAGGTGCAGCTGGTGGAGTC |
| | Nucleic acid | TGGGGGAGGCTTGGCCCAGC |
| | sequence | CTGGGGGGTCCCTGAGACTC |
| | | TCCTGTGCAGCCTCGGGATT |
| | | CACCTTTAGTCGCTATTGGA |
| | | TGACCTGGGTCCGCCAGGCT |
| | | CCAGGGGGGAGACTGGAGTG |
| | | GGTGGCCAAGATAAAGTACG |
| | | ATGGAAGTGAGAAATACTAT |
| | | GCGGACTCTGTGAAGGGCCG |
| | | ATTCACCATCTCCAGAGACA |
| | | ACGCCAAGAACTCACTGTAT |
| | | CTGCAAATGGACAGCCTGAG |
| | | AGCCGAGGACACGGCTGTAT |
| | | ATTACTGTACGAGAGACTAT |
| | | AATAAAGACTACTGGGGCCA |
| | | GGGAACCCTGGTCACCGTCT |
| | | CCTCA |
| 33 | V48 | GAGGTGCAGCTGGTGGAGTC |
| | Nucleic acid | TGGGGGAGGCTTGGTCCAGC |
| | sequence | CTGGGGGGTCCCTTAGACTC |
| | | ACCTGTGCAGCCTCTGGATT |
| | | CACTTTTAGTAGGTATTGGA |
| | | TGACTTGGGTCCGCCAGGCT |
| | | CCAGGGAAGGGGCTGGAGTG |
| | | GGTGGCCAAAATAAGACACG |
| | | ATGGAGGTGAGAAATACTAT |
| | | CCGGACTCTGTGAAGGGCCG |
| | | ATTCACCGTCTCCAGAGACA |
| | | ACGCCAAGAATTCACTGTAT |
| | | CTACAAATGGACAACCTGAG |
| | | AGCCGAGGACACGGCTATGT |
| | | ATTACTGTACGAGAGACTAC |
| | | AATAAGGACCTTTGGGGCCA |
| | | GGGAACACTGGTCACCGTCT |
| | | CCTCA |
| 34 | V51 | GAGGTGCAGCTGGTGGAGTC |
| | Nucleic acid | TGGGGGAGGCTTGGTCCAGC |
| | sequence | CTGGGGGGTCCCTGAGACTC |
| | | TCCTGTGCAGCCTCTGGATT |
| | | CACCTTTAGTAGGTATTGGA |
| | | TGACCTGGGTCCGCCAGGCT |
| | | CCAGGGAAGGGGCTGGAATG |
| | | GGTGGCCAAGATAAGACACG |
| | | ATGGAGGTGAGAAATATTAT |
| | | GCGGACTCTGTGAAGGGCCG |
| | | ATTCACCATCTCCAGAGACA |
| | | ACGCCAAGAATTCACTATAT |
| | | CTACAAATGAACAGTCTGAG |
| | | AGCCGAAGACACGGCTGTGT |
| | | ATTATTGTACGAGAGACTAC |
| | | AATAAAGACTACTGGGGCCA |
| | | GGGAACCCTGGTCACCGTCT |
| | | CCTCA |

### EQUIVALENTS

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

We claim:

1. A guanylyl cyclase C (GCC) binding agent comprising:
a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16);
a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17);
a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18);
a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19) or
a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

2. The GCC binding agent of claim 1, comprising
an immunoglobulin heavy chain variable (V_{H}) region comprising an amino acid sequence that is at least 90% identical to SEQ ID NO: 1 or SEQ ID NO: 20;
an immunoglobulin heavy chain variable (V_{H}) region comprising an amino acid sequence that is at least 90% identical to SEQ ID NO; 21;
an immunoglobulin heavy chain variable (V_{H}) region comprising an amino acid sequence that is at least 90% identical to SEQ ID NO; 26;
an immunoglobulin heavy chain variable (V_{H}) region comprising an amino acid sequence that is at least 90° % identical to SEQ ID NO; 27; or
an immunoglobulin heavy chain variable (V_{H}) region comprising an amino acid sequence that is at least 90% identical to SEQ ID NO; 28.

3. A guanylyl cyclase C (GCC) binding agent comprising:
an immunoglobulin heavy chain variable (V_{H}) region comprising an amino acid sequence that is at least 90% identical to SEQ ID NO: 1 or SEQ ID NO: 20;
an immunoglobulin heavy chain variable (V_{H}) region comprising an amino acid sequence that is at least 90% identical to SEQ ID NO: 21;
an immunoglobulin heavy chain variable (V_{H}) region comprising an amino acid sequence that is at least 90% identical to SEQ ID NO: 26;
an immunoglobulin heavy chain variable (V_{H}) region comprising an amino acid sequence that is at least 90% identical to SEQ ID NO: 27; or
an immunoglobulin heavy chain variable (V_{H}) region comprising an amino acid sequence that is at least 90% identical to SEQ ID NO: 28.

4. The GCC binding agent of any one of the preceding claims, wherein the V_{H} region comprises an amino acid sequence that is at least 95% identical to any one of SEQ ID Nos:1, 20, 21, 26, 27, or 28.

5. The GCC binding agent of any one of the preceding claims, wherein the V_{H} region comprises an amino acid sequence that is identical to any one of SEQ ID NOs:1, 20, 21, 26, 27, or 28.

6. The GCC binding agent of any one of the preceding claims, wherein the GCC binding agent is selected from the group consisting of an IgA antibody, IgG antibody, IgE antibody, IgM antibody, bi- or multi-specific antibody, Fab fragment, Fab′ fragment, F(ab′)2 fragment, Fd′ fragment, Fd fragment, isolated CDRs or sets thereof; single-chain variable fragment (scFv), polypeptide-Fc fusion, single domain antibody (sdAb), camelid antibody: masked antibody, Small Modular ImmunoPharmaceuticals (“SMIPs™”), single chain, Tandem diabody, VHHs, Anticalin, Nanobody, humabody, minibodies, BiTE, ankyrin repeat protein, DARPIN, Avimer, DART, TCR-like antibody, Adnectin, Affilin, Trans-body; Affibody, TrimerX, MicroProtein, Fynomer, Centyrin; and KALBITOR.

7. The GCC binding agent of any one of the preceding claims, wherein the GCC binding agent is a single domain antibody (sdAb).

8. The GCC binding agent of any one of the preceding claims, wherein the GCC binding agent is a heavy chain only antibody.

9. The GCC binding agent of any one of the preceding claims, wherein the binding agent binds GCC with a K_{D} between about 0.3 nanomolar (nM) and about 10 nM.

10. The GCC binding agent of any one of the preceding claims, wherein the binding agent binds GCC on target cells with an EC₅₀ between about 0.5 nM and about 8 nM.

11. A method of treating a cancer comprising administering the GCC binding agent of any one of the preceding claims to a subject in need of treatment.

12. The method of claim 11, wherein the cancer is selected from gastrointestinal cancer, colorectal cancer, colorectal adenocarcinoma, colorectal leiomyosarcoma, colorectal lymphoma, colorectal melanoma, a colorectal neuroendocrine tumor, metastatic colon cancer, stomach cancer, gastric adenocarcinoma, gastric lymphoma, gastric sarcoma, esophageal cancer, squamous cell carcinoma, adenocarcinoma of the esophagus, or pancreatic cancer.

13. The method of claim 11, wherein the cancer is a gastrointestinal cancer.

14. The method of claim 13, wherein the gastrointestinal cancer is colon cancer, colorectal cancer, stomach cancer, or esophageal cancer.

15. A pharmaceutical composition comprising a GCC binding agent and a pharmaceutically acceptable carrier, wherein the GCC binding agent comprises:
a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16);
a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17);
a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18);
a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19) or
a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

16. A method of treating a cancer comprising administering an GCC binding agent to a subject in need of treatment, wherein the GCC binding agent comprises:
a heavy chain variable region (V_{H}) with complementarity determining region (CDR) sequences of HYYWS (HCDR1) (SEQ ID NO: 8), RIYPSGSTSYNPSLKS (HCDR2) (SEQ ID NO: 11) and DRSTGWSEWNSDL (HCDR3) (SEQ ID NO: 16);
a heavy chain variable region (V_{H}) with complementarity determining region (CDR) sequences of RYWMS (HCDR1) (SEQ ID NO: 9), KIRHDGGEKYYVDSVKG (HCDR2) (SEQ ID NO: 12) and DYTRDV (HCDR3) (SEQ ID NO: 17);
a heavy chain variable region (V_{H}) with complementarity determining region (CDR) sequences of RYWMT (HCDR1) (SEQ ID NO: 10), KIKYDGSEKYYADSVKG (HCDR2) (SEQ ID NO: 13) and DYNKDY (HCDR3) (SEQ ID NO: 18);
a heavy chain variable region (V_{H}) with complementarity determining region (CDR) sequences of RYWMT (HCDR1) (SEQ ID NO: 10), KIRHDGGEKYYPDSVKG (HCDR2) (SEQ ID NO: 14) and DYNKDL (HCDR3) (SEQ ID NO: 19) or
a heavy chain variable region (V_{H}) with complementarity determining region (CDR) sequences of RYWMT (HCDR1) (SEQ ID NO: 10), KIRHDGGEKYYADSVKG (HCDR2) (SEQ ID NO: 15) and DYNKDY (HCDR3) (SEQ ID NO: 18).

17. A nucleic acid encoding a VH amino acid sequence that is identical to any one of SEQ ID Nos: 1, 20, 21, 26, 27, or 28.

18. A vector comprising the isolated nucleic acid sequence of claim 17.

19. An isolated cell comprising the vector of claim 18.

20. An anti-guanylyl cyclase C (GCC) chimeric antigen receptor (CAR), wherein the anti-GCC CAR comprises an anti-GCC binding agent of any one of claims 1-10.

21. A method of inducing an immune response comprising
contacting cells with an anti-guanylyl cyclase C (GCC) chimeric antigen receptor (CAR), wherein the anti-GCC CAR comprises an anti-GCC binding agent of any one of claims 1-10.

22. A method of inducing cytotoxicity comprising
contacting cells with an anti-guanylyl cyclase C (GCC) chimeric antigen receptor (CAR), wherein the anti-GCC CAR comprises an anti-GCC binding agent of any one of claims 1-10.

23. A method of detecting the presence of cancer in a mammal, comprising:
(a) contacting a sample comprising one or more cells from the mammal with the anti-GCC binding agent of any one of claims 1-10, thereby forming a complex, and
(b) detecting the complex, wherein detection of the complex is indicative of the presence of cancer in the mammal.

24. The method of claim 23, wherein the contacting is in vitro or in vivo with respect to the mammal.

25. The method of claim 24, wherein the contacting is in vitro.


SEQUENCE LISTING (36 sequences)

SEQ ID NO: 1 - protein, 121 aa, Artificial Sequence
  source: /note=“Description of Artificial Sequence Synthetic polypeptide”
QVQLQESGPGLVKPSETLSLTCTVSGASISHYYWSWFRQPAGKGLEWIGRIYPSGSTSYNPSLKSRVAMSVDTPKNQFSLNLSSVTAADTAVYYCARDRSTGWSEWNSDLWGRGTLVTVSS

SEQ ID NO: 2 - protein, 19 aa, Artificial Sequence
  source: /note=“Description of Artificial Sequence Synthetic peptide”
MEFGLSWVFLVAIIKGVQC

SEQ ID NO: 3 - protein, 22 aa, Artificial Sequence
  source: /note=“Description of Artificial Sequence Synthetic peptide”
GSGATNFSLLKQAGDVEENPGP

SEQ ID NO: 4 - protein, 21 aa, Artificial Sequence
  source: /note=“Description of Artificial Sequence Synthetic peptide”
MALPVTALLLPLALLLHAARP

SEQ ID NO: 5 - protein, 1073 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 6 - protein, 19 aa, Artificial Sequence
  source: /note=“Description of Artificial Sequence Synthetic peptide”
MKHLWFFLLLVAAPRWVLS

SEQ ID NO: 7 - protein, 19 aa, Artificial Sequence
  source: /note=“Description of Artificial Sequence Synthetic peptide”
MELGLSWVFLVAILEGVQC

SEQ ID NO: 8 - protein, 5 aa, Artificial Sequence
  source: /note=“Description of Artificial Sequence Synthetic peptide”
HYYWS

SEQ ID NO: 9 - protein, 5 aa, Artificial Sequence
  source: /note=“Description of Artificial Sequence Synthetic peptide”
RYWMS

SEQ ID NO: 10 - protein, 5 aa, Artificial Sequence
  source: /note=“Description of Artificial Sequence Synthetic peptide”
RYWMT

SEQ ID NO: 11 - protein, 16 aa, Artificial Sequence
  source: /note=“Description of Artificial Sequence Synthetic peptide”
RIYPSGSTSYNPSLKS

SEQ ID NO: 12 - protein, 17 aa, Artificial Sequence
  source: /note=“Description of Artificial Sequence Synthetic peptide”
KIRHDGGEKYYVDSVKG

SEQ ID NO: 13 - protein, 17 aa, Artificial Sequence
  source: /note=“Description of Artificial Sequence Synthetic peptide”
KIKYDGSEKYYADSVKG

SEQ ID NO: 14 - protein, 17 aa, Artificial Sequence
  source: /note=“Description of Artificial Sequence Synthetic peptide”
KIRHDGGEKYYPDSVKG

SEQ ID NO: 15 - protein, 17 aa, Artificial Sequence
  source: /note=“Description of Artificial Sequence Synthetic peptide”
KIRHDGGEKYYADSVKG

SEQ ID NO: 16 - protein, 13 aa, Artificial Sequence
  source: /note=“Description of Artificial Sequence Synthetic peptide”
DRSTGWSEWNSDL

SEQ ID NO: 17 - protein, 6 aa, Artificial Sequence
  source: /note=“Description of Artificial Sequence Synthetic peptide”
DYTRDV

SEQ ID NO: 18 - protein, 6 aa, Artificial Sequence
  source: /note=“Description of Artificial Sequence Synthetic peptide”
DYNKDY

SEQ ID NO: 19 - protein, 6 aa, Artificial Sequence
  source: /note=“Description of Artificial Sequence Synthetic peptide”
DYNKDL

SEQ ID NO: 20 - protein, 121 aa, Artificial Sequence
  source: /note=“Description of Artificial Sequence Synthetic polypeptide”
EVQLQESGPGLVKPSETLSLTCTVSGASISHYYWSWFRQPAGKGLEWIGRIYPSGSTSYNPSLKSRVAMSVDTPKNQFSLKLSSVTAADTAVYYCARDRSTGWSEWNSDLWGRGTLVTVSS

SEQ ID NO: 21 - protein, 115 aa, Artificial Sequence
  source: /note=“Description of Artificial Sequence Synthetic polypeptide”
QVQLVESGGGLVQPGGSLRLSCTASGFTFSRYWMSWVRQAPGKGLEWVAKIRHDGGEKYYVDSVKGRFTISRDNAKNSLYLQMNSLRAEDTAVYYCATDYTRDVWGQGTAVTVSS

SEQ ID NO: 22 - protein, 115 aa, Artificial Sequence
  source: /note=“Description of Artificial Sequence Synthetic polypeptide”
QVQLVESGGGLVQPGGSLRLSCAASGFTFSRYWMSWVRQAPGKGLEWVAKIKYDGSEKYYVDSVKGRFTISRDNAKNSVYLQMNSLRAEDTGVYYCATDFTRDVWGQGTTVTVSS

SEQ ID NO: 23 - protein, 115 aa, Artificial Sequence
  source: /note=“Description of Artificial Sequence Synthetic polypeptide”
EVQLVESGGGLVQPGGSLRLSCAASGFTFSRYWMTWVRQAPGRGLEWVAKIRYDGGEKYYVDSVKGRFTISRDNAKNSLYLQMNSLRAEDTAVYYCATDFTRDVWGQGTTVTVSS

SEQ ID NO: 24 - protein, 115 aa, Artificial Sequence
  source: /note=“Description of Artificial Sequence Synthetic polypeptide”
QVQLVESGGGLVQPGGSLRLSCAASGFNFGRYWMSWVRQAPGKGREWVAKIKYDGSEKYYVDSVKGRFTISRDNAKNSLYLQMNSLRAEDTAVYYCATDFTRDVWGQGTTVTVSS

SEQ ID NO: 25 - protein, 115 aa, Artificial Sequence
  source: /note=“Description of Artificial Sequence Synthetic polypeptide”
QVQLVESGGGVVRPGGSLRLSCAASGFTFSRYWMSWVRQAPGKGREWVAKIKYDGSEKYYADSVKGRFTISRDNAKNSLYLQMNSLRADDTAVYYCATDFTRDVWGQGTTVTVSS

SEQ ID NO: 26 - protein, 115 aa, Artificial Sequence
  source: /note=“Description of Artificial Sequence Synthetic polypeptide”
EVQLVESGGGLAQPGGSLRLSCAASGFTFSRYWMTWVRQAPGGRLEWVAKIKYDGSEKYYADSVKGRFTISRDNAKNSLYLQMDSLRAEDTAVYYCTRDYNKDYWGQGTLVTVSS

SEQ ID NO: 27 - protein, 115 aa, Artificial Sequence
  source: /note=“Description of Artificial Sequence Synthetic polypeptide”
EVQLVESGGGLVQPGGSLRLTCAASGFTFSRYWMTWVRQAPGKGLEWVAKIRHDGGEKYYPDSVKGRFTVSRDNAKNSLYLQMDNLRAEDTAMYYCTRDYNKDLWGQGTLVTVSS

SEQ ID NO: 28 - protein, 115 aa, Artificial Sequence
  source: /note=“Description of Artificial Sequence Synthetic polypeptide”
EVQLVESGGGLVQPGGSLRLSCAASGFTFSRYWMTWVRQAPGKGLEWVAKIRHDGGEKYYADSVKGRFTISRDNAKNSLYLQMNSLRAEDTAVYYCTRDYNKDYWGQGTLVTVSS

SEQ ID NO: 29 - protein, 18 aa, Escherichia coli
NTFYCCELCCNPACAGCY

SEQ ID NO: 30 - DNA, 342 bp, Artificial Sequence
  source: /note=“Description of Artificial Sequence Synthetic polynucleotide”
gaggtgcagctggtggagtctgggggaggcttggtccagccgggggggtccctgagactctcctgtgcagcctctggattcacctttaatagttattggatgagttgggaccgccaggctccagggaagggcctggagtgggtggccaacataaaccaagatggaagtgagaaatactatggggactctgtgaggggccgattcaccatctccagagacaacgccaagaacacagtgtatctgcaaatgaacagcctgagagccgaggacacggctgtgtattactgtgtgagaaggagccacggcgtccgggggcaagggaccacggtcaccgtctcctca

SEQ ID NO: 31 - DNA, 345 bp, Artificial Sequence
  source: /note=“Description of Artificial Sequence Synthetic polynucleotide”
caggtgcagctggtggagtctgggggaggcttggtacagcctggggggtccctgagactctcctgtacagcctctggattcacctttagtcggtattggatgagctgggtccgccaggctccagggaaggggctggagtgggtggccaagataaggcacgatggaggtgagaaatactatgtggactctgtgaagggccgattcaccatctccagagacaacgccaagaattcactgtatctgcaaatgaacagcctgagagccgaggacacggctgtgtattactgtgcgacagactatacgagggacgtctggggccaagggaccgcggtcaccgtctcctca

SEQ ID NO: 32 - DNA, 345 bp, Artificial Sequence
  source: /note=“Description of Artificial Sequence Synthetic polynucleotide”
gaggtgcagctggtggagtctgggggaggcttggcccagcctggggggtccctgagactctcctgtgcagcctcgggattcacctttagtcgctattggatgacctgggtccgccaggctccaggggggagactggagtgggtggccaagataaagtacgatggaagtgagaaatactatgcggactctgtgaagggccgattcaccatctccagagacaacgccaagaactcactgtatctgcaaatggacagcctgagagccgaggacacggctgtatattactgtacgagagactataataaagactactggggccagggaaccctggtcaccgtctcctca

SEQ ID NO: 33 - DNA, 345 bp, Artificial Sequence
  source: /note=“Description of Artificial Sequence Synthetic polynucleotide”
gaggtgcagctggtggagtctgggggaggcttggtccagcctggggggtcccttagactcacctgtgcagcctctggattcacttttagtaggtattggatgacttgggtccgccaggctccagggaaggggctggagtgggtggccaaaataagacacgatggaggtgagaaatactatccggactctgtgaagggccgattcaccgtctccagagacaacgccaagaattcactgtatctacaaatggacaacctgagagccgaggacacggctatgtattactgtacgagagactacaataaggacctttggggccagggaacactggtcaccgtctcctca

SEQ ID NO: 34 - DNA, 345 bp, Artificial Sequence
  source: /note=“Description of Artificial Sequence Synthetic polynucleotide”
gaggtgcagctggtggagtctgggggaggcttggtccagcctggggggtccctgagactctcctgtgcagcctctggattcacctttagtaggtattggatgacctgggtccgccaggctccagggaaggggctggaatgggtggccaagataagacacgatggaggtgagaaatattatgcggactctgtgaagggccgattcaccatctccagagacaacgccaagaattcactatatctacaaatgaacagtctgagagccgaagacacggctgtgtattattgtacgagagactacaataaagactactggggccagggaaccctggtcaccgtctcctca

SEQ ID NO: 35 - protein, 357 aa, Artificial Sequence
  source: /note=“Description of Artificial Sequence Synthetic polypeptide”
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 36 - protein, 30 aa, Artificial Sequence
  source: /note=“Description of Artificial Sequence Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS